(19) United States
(12) Patent Application Publication
(10) Pub. No.: US 2025/0305923 A1
(43) Pub. Date: Oct. 2, 2025

(54) DEVICES AND METHODS EMPLOYING DAMPING OF VIBRATION IN FLUIDS

(71) Applicant: HYDRAMOTION LIMITED, Malton, North Yorkshire, Yorkshire (GB)

(72) Inventor: John Gallagher, Malton, North Yorkshire, Yorkshire (GB)

(21) Appl. No.: 18/869,989

(22) PCT Filed: May 26, 2023
(86) PCT No.: PCT/GB2023/051404

§ 371 Date: Nov. 27, 2024

(30) Foreign Application Priority Data
May 27, 2022 (GB) 2207881.0

(51) Int. Cl. G01N 11/16

(52) U.S. Cl. CPC G01N 11/16

(57) ABSTRACT

Determining a physical property of a fluid by: vibrating a vibratory transducer element in a fluid at a vibration frequency, wherein the vibratory transducer element comprises a fluid-contacting elongate member characterised by a width, a half width that is equal to half of the width, and a length that is greater than the width, wherein the half width is less than a propagation depth of a shear wave in the fluid at the vibration frequency; making a measurement of the vibration of the vibratory transducer element in the fluid at the vibration frequency; and determining, based on the measurement of the vibration, a physical property of the fluid such as a viscosity, a viscoelasticity, a density, a fluid stiffness, a loss tangent, a storage modulus, a loss modulus, or a yield stress.

### TECHNICAL FIELD

This disclosure relates to the damping of vibrations within fluids, including the use of damping to obtain measurements of physical and rheological properties of materials such as measurements of viscosity.

### BACKGROUND

Physical and rheological properties of a fluid can be measured by applying an oscillatory stimulus to the fluid and observing a fluid mechanical response. From an observed fluid mechanical response (for example, a degree of damping and/or stiffness, and/or a resonant frequency), a measurement of a property of a fluid can be obtained, such as viscosity, density, storage modulus, loss modulus, and loss tangent.

By way of example, a degree of damping may be determined from an amplitude of vibration or a change in amplitude, a resonant frequency or a change in resonant frequency, a rate of decay of vibration, or a quality (Q) factor or a loss factor, wherein a loss factor is a reciprocal of a quality factor.

### SUMMARY

In accordance with the techniques of this disclosure, geometric damping is employed to provide damping of vibrations of a body vibrating in a fluid and/or to determine properties of the fluid, such as a viscosity, a viscoelasticity, a density, a fluid stiffness, a loss tangent, a storage modulus, a loss modulus, and a yield stress. As discussed in more detail below, geometric damping may be achieved using an elongate member vibrating in the fluid, the elongate member having a half width that is relatively smaller than a viscoelastic propagation depth of a shear wave in the fluid at the frequency of vibration. The elongate member may be cylindrical (in which case the half width is equal to the radius of the cylinder), but it is not required to be cylindrical.

By employing geometric damping, improved linearity may be obtained. In any measurement scenario, the quality and integrity of the measurement is improved if that measurement does not require any compensation for non-linearity, such as an algorithm that might be needed to modify a measured output to compensate for a non-linear deviation of the output against the measured variable. As well as simplicity, linearity may provide improved accuracy through not requiring non-linear correction which may introduce errors due to an imperfect compensation algorithm. In addition, by employing geometric damping, a greater independence from secondary variables may be obtained. For example, a damping-related measurement may be dependent on viscosity and not density, elasticity or frequency. Therefore variations in any of density, elasticity or frequency, which might be expected to occur in a real-world measuring situation, do not affect a measurement of viscosity. There may be no need to separately measure all of these factors and compensate for changes in them when determining, for example, the viscosity.

According to a first aspect there is provided a method of determining a physical property of a fluid, the method comprising: vibrating a vibratory transducer element in a fluid at a vibration frequency, wherein the vibratory transducer element comprises a fluid-contacting elongate member characterised by a width, a half width that is equal to half of the width, and a length that greater than the width, wherein the half width is less than a propagation depth of a shear wave in the fluid at the vibration frequency; making a measurement of the vibration of the vibratory transducer element in the fluid at the vibration frequency; and determining a physical property of the fluid based on the measurement of the vibration.

In some embodiments, determining the physical property of the fluid based on the measurement of vibration comprises determining one or more of: a viscosity, a viscoelasticity, a density, a fluid stiffness, a loss tangent, a storage modulus, a loss modulus, and a yield stress.

In some embodiments, making the measurement of the vibration comprises determining a first quantity indicative of a degree of damping of the vibratory transducer element in the fluid at the vibration frequency, wherein the method further comprising vibrating the vibratory transducer element in the fluid at a further vibration frequency and determining a second quantity indicative of a degree of damping of the vibratory transducer element in the fluid at the further vibration frequency, wherein determining the physical property of the fluid based on the measurement of the vibration comprises determining a viscoelasticity of the fluid based on the quantities indicative of the degree of damping at the vibration frequency and at the further vibration frequency.

In some embodiments, making the measurement of the vibration comprises determining a resonant frequency of the vibratory transducer element in the fluid, wherein determining the physical property of the fluid based on the measurement of the vibration comprises determining a density of the fluid based on the resonant frequency.

In some embodiments, the propagation depth is a distance over which an amplitude of a shear wave propagating in the fluid at the vibration frequency is reduced by a factor of 1/e, wherein e is the base of natural logarithms.

In some embodiments, the propagation depth of a shear wave propagating in the fluid at the vibration frequency is given by the expression:

$$\frac{1}{sin \frac{Δ}{2}} \sqrt{\frac{2 μ}{ρω sin Δ}} , \qquad ( Equation 1 )$$

wherein μ is a viscosity of the fluid, ρ is a density of the fluid, ω is the angular frequency of vibration, and Δ varies between 0 and π/2 (in radians) and is defined by the loss tangent, tan Δ, and wherein tan Δ is equal to the following expression, in which G′ is a storage modulus of the fluid:

$$\frac{μω}{{G}^{′}} . \qquad ( Equation 2 )$$

In some embodiments, the half width of the elongate member is less than 75% of the propagation depth, optionally less than 60%, optionally less than 50%, optionally less than 40%, optionally less than 25%, optionally less than 10%, optionally less than 5%, optionally less than 2%, optionally less than 1%, or optionally less than 0.5%.

In some embodiments, the elongate member has a substantially or wholly circular cross section along 50%, 70%, 90% or 100% of its length. Optionally, along 50%, 70%, 90% or 100% of its length, the elongate member has a cross section that has a circularity in the range 0.75 to 1, optionally in the range 0.8 to 1, optionally in the range 0.85 to 1, optionally in the range 0.9 to 1, optionally in the range 0.95 to 1, more preferably in the range 0.9 to 1, optionally in the range 0.95 to 1, wherein the circularity of a cross section shape is calculated by 4πA/p², where A is the convex area of the cross section shape and p is the convex perimeter of the cross section shape.

In some embodiments, a half width of the elongate member calculated at a point along its length is based on the convex perimeter or the convex area of the cross section of the elongate member at that point along its length. Optionally, the half width is calculated based on the convex perimeter of the shape of the cross section by the expression p/2π. Alternatively, the half width may be calculated based on the convex area of the shape of the cross section by the expression √(A/π). If the elongate member has a circular cross section then both of these expressions produce the radius of the circle and so the half width of a circular cross section is the radius of the circle.

In some embodiments, the elongate member has a constant cross section along more than 50%, more than 60%, more than 70%, more than 80%, more than 90% or 100% of its length.

In some embodiments, the elongate member only has a constant cross section along less than 50%, less than 40%, less than 30%, less than 20%, less than 10% of its length, or the cross section varies continuously along its length.

In some embodiments, the area of the cross section increases or decreases monotonically along the length of the elongate member.

In some embodiments, the elongate member is straight.

In some embodiments, the elongate member is axially symmetric along its length.

In some embodiments, the elongate member is non-straight. For example, the elongate member may comprise a closed loop.

In some embodiments, the elongate member comprises one of: a circular cylinder, a cone, a frustrum of a cone, a torus, and an arcuate portion of a torus.

In some embodiments, the half width the elongate member that is less than the propagation depth is a maximum half width along the length of the elongate member.

In some embodiments, the half width of the elongate member that is less than the propagation depth is an average half width along the length of the elongate member. Optionally, the average half width is calculated as an arithmetic mean of the half width along the length of the elongate member or is an average half width that is calculated as twice a volume of the elongate member divided by the surface area of the elongate member.

In some embodiments, the width of the elongate member is greater than 0.5 mm, and/or greater than 1 mm, and/or greater than 2 mm, and/or greater than 5 mm, and/or greater than 10 mm, and/or greater than 20 mm, and/or greater than 50 mm.

In some embodiments, the length of the elongate member is greater than a multiple of the half width of the elongate member (the half width being half of the width of the elongate member), and wherein the multiple is one of: 2, 3, 4, 5, 6, 7, 8, 9, 10, 11, 12, 13, 14, 15, 16, 17, 18, 19, 20, 25, 30, 35, 40, 45, or 50. Expressed another way, the length of the elongate member may be greater than a multiple of the width of the elongate member, wherein the multiple is one of 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, and 25.

In some embodiments, a viscosity of the fluid is greater than 100 Pa·s, and/or or greater than 200 Pas, and/or greater than 500 Pa·s.

In some embodiments, a density of the fluid is between 500 kg/m³ and 1500 kg/m³, and/or between 700 kg/m³ and 1300 kg/m³, and/or between 900 kg/m³ and 1100 kg/m³.

In some embodiments, the frequency of vibration is less than 10 kHz, and/or less than 7 kHz, and/or less than 5 kHz, and/or less than 3 kHz, and/or less than 2 kHz, and/or less than 1 kHz, and/or less than 500 Hz.

In some embodiments, the width of the elongate member is between 1 nm and 500 nm. Such embodiments may be described as ‘nanoscale’ or nanoscopic-scale embodiments. In some other embodiments, the width of the elongate member is between 500 nm and 500 μm. Such embodiments may be described as ‘microscale’ or microscopic-scale embodiments. An appropriately dimensioned device, which may be a nanoscale or microscale device, May vibrate at a low frequency to advantageously measure fluid properties of fluids with low viscosities, such as less than 1 mPa·s, or may vibrate at a high frequency to advantageously measure fluid properties of fluids with low viscosities, such as less than 1 mPa·s because, at such small scales, the width of the elongate member may still be small relative to the propagation depth at such high frequencies.

In some embodiments, a viscosity of the fluid may be less than 100 Pas, less than 10 Pas, less than 1 Pa·s, less than 100 mPa·s, less than 10 mPa·s, or less than 1 mPa·s. At any of such viscosities, the frequency of vibration may be greater than 500 Hz, greater than 1 kHz, greater than 2 kHz, greater than 3 kHz, than 4 kHz, greater than 5 kHz, greater than 7 kHz, and/or greater than 10 KHz.

In some embodiments the fluid is a Newtonian fluid. In other embodiments the fluid is a non-Newtonian fluid, such as a viscoelastic fluid or a yield stress fluid.

In some embodiments, the vibratory transducer element comprises a shaft that has a longitudinal axis, wherein the elongate member is connected to the shaft and wherein the elongate member is not collinear with the longitudinal axis of the shaft. Optionally, during vibration of the vibratory transducer element at the vibration frequency, the flow of fluid around the elongate member is laminar flow. Alternatively or additionally, a Reynolds number, Re, of fluid flow around the elongate member is less than one, wherein the Reynolds number is equal to 2 R v ρ/μ, where μ is a viscosity of the fluid, ρ is a density of the fluid, R is the half width of the elongate member, and v is a maximum (vibrational) velocity of the elongate member relative to the fluid during vibration of the vibratory transducer, wherein optionally the Reynolds number is less than 1000, or less than 300, or less than 100, or less than 30, or less than 10, or less than 3, or less than 1, or less than 0.9, or less than 0.8, or less than 0.75, or less than 0.7, or less than 0.6, or less than 0.5, or less than 0.4, or less than 0.3, or less than 0.25, or less than 0.2, or less than 0.1. Alternatively or additionally, the elongate member may have a first end and a second end, wherein one or both of the first and second end is spaced from the longitudinal axis of the shaft by an offset distance that is greater than the half width of the elongate member, wherein, optionally, the offset distance is greater than a multiple of the width and the multiple is 2, 3, 4, 5, 6, 7, 8, 9, 10, 11, 12, 13, 14, 15, 20, 30 or 50. Alternatively or additionally, the vibratory transducer element may comprise a plurality of elongate members connected to the shaft that are each not collinear with the axis of the shaft, each having a half width that is less than the propagation depth of a shear wave in the fluid at the vibration frequency, and wherein, optionally, the half width of a first elongate member of the plurality of elongate members is different from the half width of a second elongate member of the plurality of elongate members, and wherein, optionally, two, three, four, five or more of the elongate members of the plurality of elongate members may have uniquely different half widths. Alternatively or additionally, the elongate member may comprise a first end and a second end, wherein the elongate member is connected to the shaft at the first end and optionally also at the second end. Alternatively or additionally, the shaft may comprise a bob and the elongate member may be connected to the shaft at the bob.

In some embodiments, vibrating the vibratory transducer element comprises vibrating the vibratory transducer element with an oscillatory rotational motion. Optionally, the elongate member may be straight and vibrating the transducer element may comprise vibrating the elongate member with an oscillatory rotational motion about an axis along the length of the elongate member.

In some embodiments, vibrating the vibratory transducer may comprise vibrating the vibratory transducer with an oscillatory rectilinear or curvilinear motion. Optionally, where the vibratory transducer comprises a shaft having a longitudinal axis and the elongate member is connected to the shaft but not collinear with the longitudinal axis of the shaft, vibrating the vibratory transducer with an oscillatory rectilinear or curvilinear motion may comprise vibrating the shaft with an oscillatory rotary, rectilinear or curvilinear motion that causes the elongate member to move in an oscillatory rectilinear or curvilinear motion.

According to a further aspect there is provided a method of determining a property of a fluid, the method comprising: vibrating a vibratory transducer element in a fluid at a frequency of vibration firstly at a first amplitude of vibration and secondly at a second amplitude of vibration, wherein the vibratory transducer element comprises a fluid-contacting elongate member characterised by a width, a half width that is equal to half of the width, and a length that is greater than the width; determining a first quantity indicative of a degree of damping based on the vibration of the vibratory transducer element in the fluid at the first amplitude; determining a second quantity indicative of a degree of damping based on the vibration of the vibratory transducer element in the fluid at the second amplitude; determining a property of the fluid based on a difference between the first and second quantities.

In some embodiments, determining the property of the fluid comprises determining a dipolarity of the wave field around the vibratory transducer at the frequency of vibration based on the difference between the first and second quantities. Optionally, the method may further comprise determining, based on the determined dipolarity, a degree to which the half width of the elongate member is less than the propagation depth of a shear wave in the fluid at the frequency of vibration and/or determining whether or not the half width of the elongate member is less than the propagation depth of a shear wave in the fluid at the frequency of vibration or less than some predetermined fraction of the propagation depth, such as less than 50% of the propagation depth.

In some embodiments, a quantity indicative of a dipolarity of the wave field around the vibratory transducer at the frequency of vibration is not explicitly determined, and the difference between the first and second quantities indicative of a degree of damping may be used to determine whether or not the half width of the elongate member is less than the propagation depth (or some predetermined fraction of the propagation depth, such as less than 50% of the propagation depth) of a shear wave in the fluid at the frequency of vibration and/or a degree to which the half width of the elongate member is less than the propagation depth of a shear wave in the fluid at the frequency of vibration.

In some embodiments, determining the property of the fluid comprises determining a Reynolds number of the fluid based on the difference between the first and second quantities.

In some embodiments, determining the property of the fluid comprises determining, based on the difference between the first and second quantities, a velocity of vibration relative to the fluid, a viscosity of the fluid, or a density of the fluid.

In some embodiments, determining the first and second quantities indicative of a degree of damping comprises determining first and second Q factors. In other embodiments, determining the first and second quantities indicative of a degree of damping comprises determining first and second loss factors, wherein a loss factor is a reciprocal of a Q factor.

In some embodiments, the determined property of the fluid is a property of the flow of the fluid due to the vibration of the vibratory transducer element in the fluid at the frequency of vibration for one or both of the first and second amplitudes.

In some embodiments, a change in Reynolds number may be considered to be proportional to the ratio between the change in Q factor and the change in amplitude of vibration. If the ratio is zero or negligible, then the Reynolds number is low and the vibrational flow may be estimated to be laminar. If the ratio is non-zero or greater than a threshold value, then the fluid flow may not be completely laminar and the Reynolds number may have increased. The degree to which the Reynolds number has increased may be dependent on the value of the ratio.

In some embodiments, the method further comprises performing any of the above-described methods for determining a physical property of a fluid. For example, performing multiple vibratory tests at different amplitudes may indicate whether quadratic or geometric damping is present, and thus whether geometric damping is to be expected when determining the physical property of the fluid. In quadratic damping, the damping force varies quadratically with the square of the velocity. As will be discussed in more detail later, in geometric damping, the damping force varies linearly with the velocity.

According to a further aspect there is provided a device comprising: a shaft configured to vibrate at a vibration frequency, the shaft having a longitudinal axis; and an elongate member connected to the shaft but not collinear with the longitudinal axis of the shaft, the elongate member characterised by a width, a half width that is equal to half of the width, and a length that is greater than the width. The device may be configured to vibrate the shaft in a fluid such as a liquid. The fluid may be a Newtonian fluid or a non-Newtonian fluid.

In some embodiments, at least a portion of the elongate member is offset from the longitudinal axis by an offset distance that is greater than the half width of the elongate member. Optionally, the offset distance is greater than a multiple of the half width of the elongate member and the multiple is 2, 3, 4, 5, 10, 15, 20, 30 or 50. The at least a portion of the elongate member that is offset from the longitudinal axis by such an offset distance may be a portion along a longitudinal axis along the length of the elongate member extending through a centre of the elongate member.

In some embodiments, the elongate member has a first end and a second end, wherein one or both of the first and second ends is spaced from the longitudinal axis of the shaft by an offset distance that is greater than the half width. Optionally, the offset distance is greater than a multiple of the half width of the elongate member and the multiple is 2, 3, 4, 5, 10, 15, 20, 30 or 50.

In some embodiments, the elongate member has a substantially or wholly circular cross section along 50%, 70%, 90% or 100% of its length. Optionally, along 50%, 70%, 90% or 100% of its length, the elongate member has a cross section that has a circularity in the range 0.75 to 1, optionally in the range 0.8 to 1, optionally in the range 0.85 to 1, optionally in the range 0.9 to 1, optionally in the range 0.95 to 1, optionally in the range 0.98 to 1, optionally in the range 0.99 to 1, wherein the circularity of a cross section shape is calculated by 4πA/p², where A is the convex area of the cross section shape and p is the convex perimeter of the cross section shape.

In some embodiments, a half width of the elongate member is calculated at a point along its length based on the convex perimeter or the convex area of the cross section of the elongate member at that point along its length. Optionally, the half width is calculated based on the convex perimeter of the shape of the cross section by the expression p/2x. Alternatively, the half width may be is calculated based on the convex area of the shape of the cross section by the expression v (A/T). If the elongate member has a circular cross section then both of these expressions produce the radius of the circle and so the half width of a circular cross section is the radius of the circle.

In some embodiments, the elongate member has a constant cross section along more than 50%, more than 60%, more than 70%, more than 80%, more than 90% or 100% of its length.

In some embodiments, the elongate member only has a constant cross section along less than 50%, less than 40%, less than 30%, less than 20%, less than 10% of its length, or the cross section varies continuously along its length.

In some embodiments, the area of the cross section increases or decreases monotonically along the length of the elongate member.

In some embodiments, the elongate member is straight.

In some embodiments, the elongate member is axially symmetric along its length.

In some embodiments, the elongate member is non-straight. For example, the elongate member may comprise a closed loop.

In some embodiments, the elongate member comprises one of: a circular cylinder, a cone, a frustrum of a cone, a torus, and an arcuate portion of a torus.

In some embodiments, the half width the elongate member that is less than the propagation depth is a maximum half width along the length of the elongate member.

In some embodiments, the half width of the elongate member that is less than the propagation depth is an average half width along the length of the elongate member. Optionally, the average half width is calculated as an arithmetic mean of the half width along the length of the elongate member or is an average half width that is calculated as twice a volume of the elongate member divided by the surface area of the elongate member.

In some embodiments, the width of the elongate member is greater than 0.5 mm, and/or greater than 1 mm, and/or greater than 2 mm, and/or greater than 5 mm, and/or greater than 10 mm, and/or greater than 20 mm, and/or greater than 50 mm.

In some embodiments, a length of the elongate member is greater than a multiple of the half width of the elongate member, and wherein the multiple is one of: 3, 4, 5, 6, 7, 8, 9, 10, 11, 12, 13, 14, 15, 16, 17, 18, 19, 20, 25, 30, 35, 40, 45 or 50.

In some embodiments, the device comprises a plurality of elongate members connected to the shaft, each having a respective width, half width and length. Optionally, the half width of a first elongate member of the plurality of elongate members is different from the half width of a second elongate member of the plurality of elongate members, and wherein, optionally, two, three, four, five or more of the elongate members of the plurality of elongate members may have uniquely different half widths.

In some embodiments, the device is configured to vibrate the shaft torsionally about its longitudinal axis and/or longitudinally along its longitudinal axis and/or transversely to its longitudinal axis.

In some embodiments the device comprises a bob. Optionally, the at least one elongate member is connected to the shaft at the bob.

In some embodiments the device is a device for determining a physical property of a fluid, the device configured to vibrate the shaft in the fluid while the elongate member is in contact with the fluid, the device configured to: make a measurement of the vibration of the shaft in the fluid; and determine a physical property of the fluid based on the measurement of the vibration. The determined physical property may be a viscosity of the fluid or a density of the fluid or a storage modulus of the fluid or a loss modulus of the fluid or a loss tangent of the fluid. Such a device may be used to implement any of the above-described methods.

According to a further aspect there is provided a system comprising a fluid and any of the above-described devices, wherein the elongate member and at least a portion of the shaft are in contact with the fluid and the shaft is configured to vibrate at a vibration frequency, wherein the half width of the elongate member is less than a propagation depth of a shear wave in the fluid at the vibration frequency. In some embodiments, the system may comprise the fluid captive in a container. In some embodiments, the fluid may be flowing in a system, such as in an open- or closed-loop conduit system, the fluid flowing past the elongate member and shaft. In some embodiments, the device may be a damper, wherein the elongate member is configured to damp vibrations such as torsional vibrations in the shaft at the vibration frequency. In this way, the shaft may be couplable or integral with a source of vibration in a machine or device and provide damping to such vibrations.

According to a further aspect, there is provided a method of controlling a damping behaviour of a shaft configured to vibrate in a fluid, the method comprising: providing a shaft configured to vibrate at a vibration frequency, the shaft having a longitudinal axis; and providing an elongate member connected to the shaft but not collinear with the longitudinal axis of the shaft, the elongate member characterised by a width, a half width that is equal to half of the width, and a length that is greater than the width, the elongate member and at least a portion of the shaft configured to vibrate in the fluid at the vibration frequency, wherein the half width of the elongate member is less than a propagation depth of a shear wave in the fluid at the vibration frequency. The propagation depth may be a distance over which an amplitude of a shear wave propagating in the fluid at the vibration frequency is reduced by a factor of 1/e, wherein e is the base of natural logarithms, and may be a viscoelastic propagation depth as described herein.

In some embodiments, the elongate member has a first end and a second end, wherein one or both of the first and second ends is spaced from the longitudinal axis of the shaft by an offset distance that is greater than the half width of the elongate member.

In some embodiments, the elongate member is provided such that, during vibration of the shaft at the vibration frequency, the flow of fluid around the elongate member is laminar flow.

In some embodiments, the elongate member is provided such that during vibration of the shaft in the fluid at the vibration frequency, a Reynolds number, Re, of fluid flow around the elongate member is less than 1000, or less than 300, or less than 100, or less than 30, or less than 10, more preferably less than 3, or less than 1, or less than 0.9, or less than 0.8, or less than 0.75, or less than 0.7, or less than 0.6, or less than 0.5, or less than 0.4, or less than 0.3, or less than 0.25, or less than 0.2, or less than 0.1, wherein the Reynolds number is given by

$$R e = \frac{2 R v ρ}{μ} ,$$

wherein μ is a viscosity of the fluid, ρ is a density of the fluid, R is the half width of the elongate member, and v is a maximum velocity of the elongate member relative to the fluid during vibration of the shaft.

In some embodiments the shaft is provided with a plurality of elongate members connected to the shaft that are each not collinear with the longitudinal axis of the shaft, each having a half width that is less than the propagation depth of a shear wave in the fluid at the vibration frequency. Optionally, the half width of a first elongate member of the plurality of elongate members may be different from the half width of a second elongate member of the plurality of elongate members.

In some embodiments, the one or more elongate members provided to the shaft are all straight, all non-straight, or a mixture of straight and non-straight elongate members.

According to a further aspect, there is provided a device comprising means for performing any of the above-described methods. The device may comprise, for each step of a method, means for performing that step. A means for performing any step of the method may be a means for performing multiple steps of the method.

According to a further aspect, there is provided a computer-readable medium having instructions stored thereon that, when executed by a processor of a vibratory transducer comprising an elongate member, such as an elongate member in accordance with the techniques described herein, cause the device to perform any of the above-described methods.

### BRIEF DESCRIPTION OF THE DRAWINGS

The invention will be described in more detail by way of example only with reference to the accompanying drawings, in which:

FIG. 1 illustrates the propagation of a shear wave in a viscous fluid from a flat plane surface;

FIG. 2 illustrates a shear wave radially propagating in a viscoelastic fluid from a curved surface;

FIG. 3 illustrates shear waves radially propagating from curved surfaces of cylinders in which one cylinder is displaced from the axis of rotation;

FIG. 4 shows a graph of measured damping factor illustrating the application of the techniques of this disclosure;

FIG. 5 illustrates a cylindrical element under lateral vibration causing a dipole wave field;

FIG. 6 illustrates laminar flow around a cylindrical element under lateral vibration;

FIGS. 7 to 31 illustrate vibratory transducers or portions thereof in accordance with the techniques of this disclosure or for use with processes in accordance with the techniques of this disclosure;

FIG. 32 illustrates schematically an example apparatus for analysing a fluid using one or more techniques of this disclosure;

FIG. 33 illustrates schematically a further example apparatus for analysing a fluid using one or more techniques of this disclosure;

FIG. 34 illustrates a flow chart of a method in accordance with the techniques of this disclosure; and

FIG. 35 illustrates a flow chart of a further method in accordance with the techniques of this disclosure.

### DETAILED DESCRIPTION

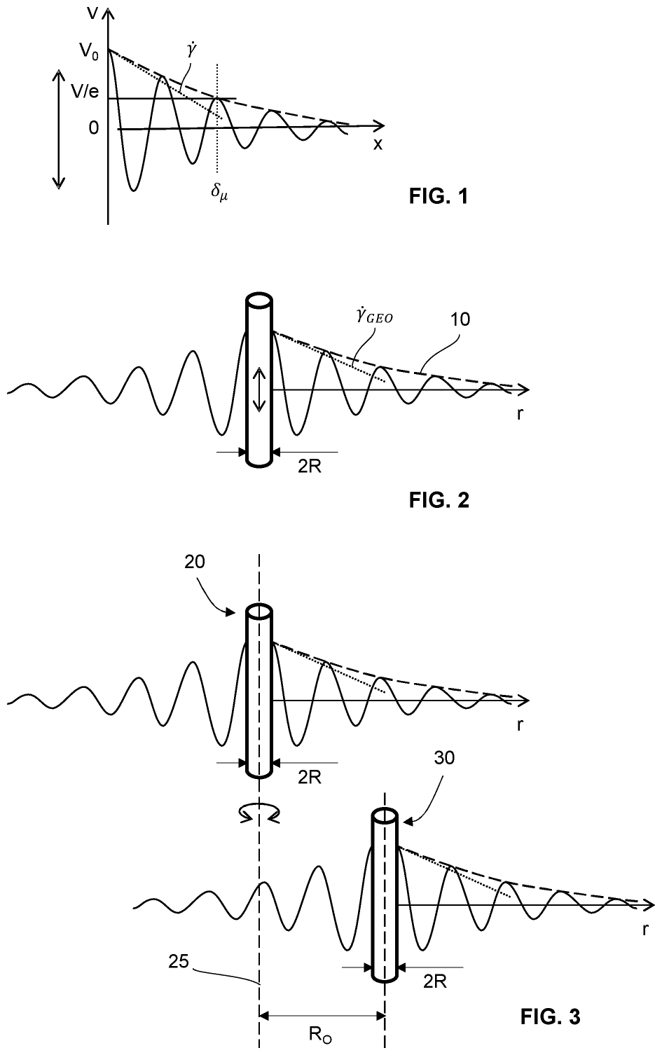

FIG. 1 illustrates the propagation of a shear wave in a viscous fluid from a flat plane surface oscillating with a surface velocity V₀. The depth at which the amplitude or velocity of the wave (shear velocity) reduces to 1/e of its value at the surface due to viscous damping is the propagation depth, δ_{μ}, given by the expression:

$${δ}_{μ} = \sqrt{\frac{2 {μ}^{′}}{ω ρ}} , \qquad ( Equation 3 )$$

where μ′ is the dynamic viscosity, p is the density of the fluid, ω is the angular frequency.

The decreasing amplitude of the wave caused by the viscosity creates a shear stress, τ_{Sμ}, at the vibrating surface that is a product of the rate of change of velocity at the surface (i.e. the shear rate \dot{γ}) and the fluid viscosity, μ′, given by the following expression:

$${τ}_{S μ} = \dot{γ} {μ}^{′} . \qquad ( Equation 4 )$$

The shear rate \dot{γ}_{μ}, at the oscillating surface caused by wave attenuation can be determined by differentiating an expression for the wave velocity with respect to the distance from the surface, and evaluating the expression at the surface, leading to the following expression:

$${\dot{γ}}_{μ} = - {V}_{0} α = - \frac{{V}_{0}}{{δ}_{μ}} = - {V}_{0} \sqrt{\frac{ω ρ}{2 {μ}^{′}}} , \qquad ( Equation 5 )$$

where V₀ is a shear velocity at the oscillating surface.

The shear rate due to viscous attenuation is proportional to the square root of the frequency and proportional to the square root of the density and proportional to the square root of the reciprocal of the viscosity.

Therefore, the shear stress at the surface (which is a product of the viscosity and shear rate at the surface) is non-linear:

$${τ}_{S μ} = - {V}_{0} \sqrt{\frac{ω ρ}{2 {μ}^{′}}} {μ}^{′} = - {V}_{0} \sqrt{\frac{{μ}^{′} ω ρ}{2}} . \qquad ( Equation 6 )$$

In addition to viscous effects, a fluid may show elastic behaviour, which is dependent on the storage modulus, G′. The presence of G′ reduces the loss tangent, tan Δ, defined by the following expression:

$$tan Δ = \frac{{G}^{″}}{{G}^{′}} = \frac{ω {μ}^{′}}{{G}^{′}} . \qquad ( Equation 7 )$$

For a purely viscous fluid, tan Δ=∞. With increased elastic behaviour the fluid becomes less lossy, allowing the wave to propagate further into the fluid. Taking elasticity into account, the propagation depth is given by the following expression:

$${δ}_{μ , {G}^{′}} = {δ}_{μ} \cdot \frac{1}{sin \frac{Δ}{2} \sqrt{2 sin Δ}} = \frac{1}{sin \frac{Δ}{2}} \sqrt{\frac{{μ}^{′}}{ρω sin Δ}} \qquad ( Equation 8 )$$

where δ_{μ,G′} is the viscoelastic propagation depth, δ_{μ} is the purely viscous propagation depth, and 1/(sin (Δ/2) √(2 sin Δ)) is a quantity that scales the propagation depth due to the fluid's elastic behaviour. This quantity is also equal to 1/√(sin Δ(1−cos Δ)).

For a purely viscous fluid, Δ is equal to 90° and so the scaling quantity given by

$$1 / ( sin ( \frac{Δ}{2} ) √ ( 2 sin Δ ) ) ( or equivalently 1 / √ ( sin Δ ( 1 - cos Δ ) ) )$$

is equal to 1 and the purely viscous propagation depth, δ_{μ}, is recovered. Thus it is appropriate to refer to a viscoelastic propagation depth using this expression even for fluids that exhibit little or no viscoelasticity. For values of Δ less than 90°, the scaling quantity is greater than 1 and so the propagation depth is increased relative to the purely viscous propagation depth.

The shear rate at the oscillating surface due to both viscosity and elasticity is given by the expression:

$${\dot{γ}}_{μ {G}^{′}} = - \frac{{V}_{0}}{{δ}_{μ , {G}^{′}}} = - {V}_{0} sin \frac{Δ}{2} \sqrt{\frac{ρω sin Δ}{μ '}} . \qquad ( Equation 9 )$$

The shear stress at the oscillating surface due to both viscosity and elasticity is given by the expression:

$${τ}_{S μ {G}^{′}} = {\dot{γ}}_{μ {G}^{′}} {μ}^{′} = - {V}_{0} sin \frac{Δ}{2} \sqrt{ρ ω {μ}^{′} sin Δ} . \qquad ( Equation 10 )$$

The shear stress is a non-linear function of the fluid viscosity, fluid density, frequency and storage modulus (via the loss tangent). As the elasticity G′ increases, tan Δ decreases, Δ decreases from a maximum of π/2, and both sin Δ and sin Δ/2 decrease, and so the damping shear stress is reduced as the elasticity G′ increases. This explains why viscoelastic fluids show reduced damping compared with Newtonian fluids of ‘similar’ viscosity.

FIG. 2 illustrates a shear wave radially propagating in a viscoelastic fluid from a curved surface with radius R. While a viscoelastic fluid is relatively lossless over a short distance, FIG. 2 shows the amplitude decreasing because the potential energy of each wave peak has to be maintained as the radial distance increases, the energy spread out over an increasing circumferential length (2πr). A line 10 of constant potential energy is shown in FIG. 2. The distribution of energy over the increasing circumferential length leads to a reduced energy per unit volume and therefore a smaller peak height. This attenuation of amplitude due to geometric considerations appears similar to damping although it does not itself dissipate energy.

The change in height causes a decrease in velocity that is proportional to 1/r and this change of velocity gives rise to a shear rate, \dot{γ}_{RAD}, which combines with the viscosity to create a shear stress, τ_{RAD}, which will have a component in phase with the surface velocity, which causes energy to be dissipated. This effect is termed ‘geometric damping’ herein.

If, instead of a planar surface oscillating as in FIG. 1, a cylindrical surface of radius R is oscillating, an expression for the velocity of a radial shear wave at a location r from the central axis of the cylinder may be obtained and itself differentiated with respect to r to obtain the radial shear rate:

$${\dot{γ}}_{r} = - {V}_{r} ( \frac{1}{r} + \sqrt{\frac{ω ρ}{μ}} {e}^{i ϕ} ) = - {V}_{r} ( \frac{1}{r} + \frac{1}{{δ}_{μ {G}^{′}}} ) \qquad ( Equation 11 )$$

where Vᵣ is a shear velocity at the surface, ϕ is a phase adjustment angle given by tan⁻¹(β/α) and equal to π/2−Δ/2, where β is the wavenumber of the propagating wave (i.e. 2π/λ, where λ is a wavelength of the propagating wave).

The shear stress at the cylindrical surface, where r=R, is then given by the expression:

$$\begin{matrix}{τ}_{S μ {G}^{′}} = {\dot{γ}}_{μ {G}^{′}} {μ}^{′} = - {V}_{r} ( \frac{1}{R} + \frac{1}{{δ}_{μ {G}^{′}}} ) {μ}^{′} \\ = - {V}_{r} ( \frac{1}{R} + \sqrt{\frac{ω ρ}{\vert μ \vert}} \cdot {e}^{i ϕ} )\end{matrix} \qquad ( Equation 12 )$$

The 1/R term is an in-phase shear gradient. The shear rate for this component is in phase with velocity for any degree of viscoelasticity. The 1/δ_{μG}, term is an out-of-phase shear gradient. The shear rate for this component is out of phase by ϕ, which depends on the degree of viscoelasticity. The phase adjustment angle represents the angle between shear stress and velocity. A shear stress that is in phase with velocity dissipates energy.

For values of R much less than δ_{μG′}, the in-phase portion dominates and the shear rate becomes less dependent or even independent of viscosity, density, frequency and storage modulus (through tan Δ/2, which is a function of G″). If 1/δ_{μG′} is negligible compared with 1/R, then the shear stress at the cylindrical surface is given by the expression:

$${τ}_{S μ {G}^{′}} = - \frac{{V}_{r} {μ}^{′}}{R} . \qquad ( Equation 13 )$$

For values of R much greater than δ_{μG′}, the out-of-phase portion dominates and the shear rate becomes increasingly dependent on non-linear functions of viscosity, density, frequency and storage modulus. If 1/R is negligible compared with 1/δ_{μG′}, then the shear stress at the cylindrical surface is given by the expression:

$${τ}_{S μ {G}^{′}} = - \frac{{V}_{r} {μ}^{′}}{{δ}_{μ {G}^{′}}} = - {V}_{r} sin \frac{Δ}{2} \sqrt{ρ ω {μ}^{′} sin Δ} . \qquad ( Equation 14 )$$

A critical value of R is at Rₒₙₛₑₜ=δ_{μG′} because this represents a cross-over point for the value of R where the shear stress due to the 1/R term becomes larger than the shear stress due to the 1/δ_{μG}, term. In accordance with the techniques described herein, this may be considered the onset of geometric damping.

The dependence of the shear stress on non-linear functions of viscosity, density, frequency and storage modulus is reduced further when R<δ_{μG′}. It may be considered that, if the cylinder radius is less than half of the viscoelastic propagation depth, then geometric damping begins to dominate, i.e. where R<Rₒₙₛₑₜ/2. In other words, R_{geo}=Rₒₙₛₑₜ/2, where R_{geo} may be understood to be a cylinder radius that defines a regime under which geometric damping can be assumed to dominate and define the damping behaviour.

For the measurement of physical properties of a fluid, parameters can be selected to provide improved linearity of fluid loading factors, such as a fluid damping factor, CF, a stiffness loading factor, K_{F}, and an inertial loading factor, J_{F}. If R=δ_{μG′}, then an expression for the fluid viscosity at which the onset of geometric damping occurs is given by:

$${μ}_{onset}^{′} = {R}^{2} ωρ \cdot {sin}^{2} \frac{Δ}{2} \cdot sin Δ = {R}^{2} 2 π f ρ \cdot {sin}^{2} \frac{Δ}{2} \cdot sin Δ . \qquad ( Equation 15 )$$

where ω=2πf.

For example, given a cylindrical element having a radius R of 2 mm vibrating in a purely viscous fluid (sin (Δ/2)√(2 sin Δ)=1) at a frequency of 5 kHz, the fluid having a density ρ of 1000 kg/m³, then an appropriate choice of fluid viscosity, in units of Pa·s, at which R=Rₒₙₛₑₜ is given by:

$${μ}_{onset}^{′} = {2}^{2} \cdot π \cdot 5000 \cdot 1000 = 62$$

For geometric damping to begin to dominate (i.e. where R_{geo}=Rₒₙₛₑₜ/2), the required viscosity is four times higher, i.e.:

$${μ}_{g e o}^{′} = {( 2 \cdot 2 )}^{2} \cdot π \cdot 5000 \cdot 1000 = 250.$$

Similarly, a radius of a vibrating element and/or a frequency of vibration can be selected to exploit geometric damping for a given working range of viscosity and density according to operational requirements.

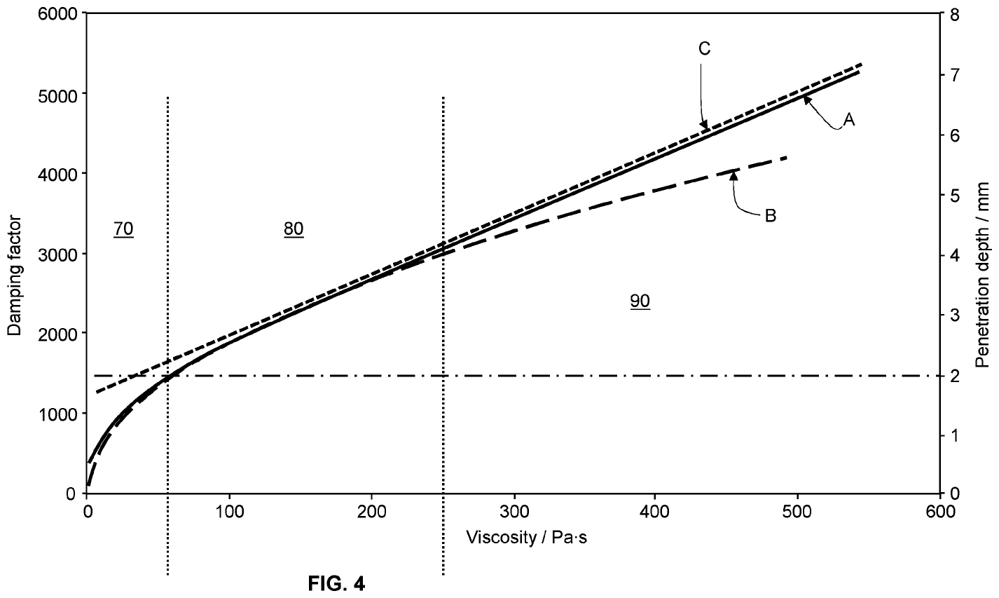

FIG. 4 shows a graph of measured damping factor for a heavy mineral oil using a vibrating cylinder of radius 2 mm at a frequency of 5 kHz, the fluid having a density ρ of 1000 kg/m³, the damping factor measured over a range of viscosity values (obtained by applying heat to the heavy mineral oil). The measured damping factor is indicated by the solid line denoted by ‘A’ in FIG. 4. The graph also shows a plot of the calculated damping factor if the damping is described by non-geometric damping, i.e. if 1/R is negligible compared with 1/δ_{μG′}. This line is denoted by ‘B’ in FIG. 4. The graph also shows a plot of the calculated damping factor if the damping is described by geometric damping, i.e. if 1/δ_{μG′}, is negligible compared with 1/R. This line is denoted by ‘C’ in FIG. 4.

FIG. 4 demonstrates that, while the viscosity is relatively low, i.e. below approximately 60 Pa·s, the radius of the vibrating element is larger than the viscous penetration depth and the shear rate is a non-linear function of viscosity. Where the viscosity is above approximately 60 Pa·s, shear wave attenuation begins to be described by geometric damping and the damping factor is seen to become increasingly linear.

FIG. 4 identifies three regions. A first region denoted by reference 70 is a non-linear region and covers viscosity values below μₒₙₛₑₜ (62 Pa·s as determined above). A second region denoted by reference 80 is a linear-transition region and covers viscosity values between μₒₙₛₑₜ and μ_{geo} (250 Pa·s as determined above). A third region denoted by reference 90 is a fully linear region and covers viscosity values above μ_{geo}. Operating in the second region 80 provides improved linearity compared with operating in the first region 70. Operating in the third region 90 provides improved linearity compared with operating in the second region 80.

Expressions for the fluid damping factor, CF, stiffness loading factor, K_{F}, and inertial loading factor, J_{F}, for a vibrating cylinder of sufficiently wide radius that geometric damping is negligible are given by the following expressions:

$${C}_{F} = ( A \cdot \frac{{R}_{G}^{2}}{{δ}_{μ , {G}^{′}}} ) {μ}^{′} = ( A \cdot {R}_{G}^{2} \frac{1}{sin \frac{Δ}{2}} \sqrt{\frac{2 {μ}^{′}}{ρ ω sin Δ}} ) {μ}^{′} \qquad ( Equation 16 )$$

$${K}_{F} = ( A \cdot \frac{{R}_{G}^{2}}{{δ}_{μ , {G}^{′}}} ) {G}^{′} = A \cdot {R}_{G}^{2} \frac{1}{sin \frac{Δ}{2}} \sqrt{\frac{2 μ '}{ρ ω sin Δ}} {G}^{′} \qquad ( Equation 17 )$$

$${J}_{F} = ( A \cdot {R}_{G}^{2} \cdot {δ}_{μ , {G}^{′}} ) ρ = ( A \cdot {R}_{G}^{2} \frac{1}{sin \frac{Δ}{2}} \sqrt{\frac{2 μ '}{ρ ω sin Δ}} ) ρ \qquad ( Equation 18 )$$

In these expressions, each of the factors has a non-linear dependency on μ′, G′, or ρ due to the presence of these terms (or quantities that are a function of these terms, such as A and its dependency on G′) inside the parentheses.

If a vibrating cylinder has a sufficiently small radius that non-geometric damping is negligible, then expressions for C_{F}, K_{F}, and J_{F} are given by:

$${C}_{F} = ( A \cdot \frac{{R}_{G}^{2}}{R} ) {μ}^{′} \qquad ( Equation 19 )$$

$${K}_{F} = ( A \cdot \frac{{R}_{G}^{2}}{R} ) {G}^{′} \qquad ( Equation 20 )$$

$${J}_{F} = ( A \cdot {R}_{G}^{2} \cdot R ) ρ \qquad ( Equation 21 )$$

It can be seen from these expressions where non-geometric damping is negligible that the fluid damping factor, C_{F}, stiffness loading factor, K_{F}, and inertial loading factor, J_{F}, no longer have any non-linear dependency on μ′, G′, or ρ. The quantities C_{F}, K_{F}, and J_{F} each respectively directly proportional to μ′, G′, and ρ, with a constant of proportionality that depends only on geometric parameters.

Improved linearity of these fluid loading factors may be advantageous. A mechanical system may have damping C, stiffness K, and inertia J. These determine the vibration frequency ω and the Q-factor of the system through the following equations:

$$ω = \sqrt{K / J} \qquad ( Equation 22 )$$

$$Q = \frac{\sqrt{K \cdot J}}{C} \qquad ( Equation 23 )$$

When the system is vibrating in air or a vacuum, these mechanical factors can be declared C₀, K₀, and J₀. When the system is vibrating a fluid, the physical properties of the fluid ‘load’ these factors by amounts C_{F}, K_{F} and J_{F} respectively.

The overall values for damping, stiffness, and inertia factors of the system taking into account the fluid loading may be expressed as:

$$C = {C}_{0} + {C}_{F} \qquad ( Equation 24 )$$

$$K = {K}_{0} + {K}_{F} \qquad ( Equation 25 )$$

$$J = {J}_{0} + {J}_{F} \qquad ( Equation 26 )$$

The overall values for C, K, and J are related to the above equations for frequency and Q-factor, which are readily measurable. The physical properties of the fluid may be determined based on the contributions of C_{F}, K_{F} and J_{F} to the vibrational behaviour. As discussed below, the techniques of this disclosure may provide simple linear relationships between C_{F}, K_{F} and J_{F} and physical properties of interest, such as density p, viscosity μ′, and storage modulus G′.

In these expressions, A is the fluid-contacting surface area of the cylindrical element and R_{G} is a ‘radius of gyration’ of the element, equal to the radius R of the cylindrical element when the cylindrical element vibrates torsionally about its axis.

In considering the effect of a moment of inertia on rotational motion of a body, the radius of gyration is as the radial distance to a point which would have a moment of inertia the same as the body's actual distribution of mass, if the total mass of the body were concentrated there. The term ‘radius of gyration’ in the present disclosure is a generalisation of this concept to consider torsional factors torsional other than moments of inertia.

In the case of the damping factor, C_{F}, the radius of gyration represents the radial distance to a point which would have the same damping effect as the body's actual damping effect if the damping were concentrated at that point.

In the case of the stiffness loading factor, K_{F}, the radius of gyration represents the radial distance to a point which would have the same stiffness loading effect as the body's actual stiffness loading effect if the stiffness loading were concentrated at that point.

In the case of the inertial loading factor, K_{F}, the radius of gyration represents the radial distance to a point which would have the same inertial loading effect as the body's actual inertial loading effect if the inertial loading were concentrated at that point.

Thus the radius of gyration defined more generally in this disclosure represents a convenient measure of the radial effect of these loading factors. The radius of gyration will be defined by the specific geometry of the cylindrical element, but will generally be assumed to have upper and lower bounds defined by a maximum and minimum radial extent of the cylindrical element from the axis of rotation, and be equal to the radius R of the cylindrical element when the cylindrical element vibrates torsionally about its axis, since all of the surface loading takes place at the cylindrical surface, all at distance R from the axis.

FIG. 3 shows a first cylinder 20 of radius R rotating torsionally about an axis 25 lengthwise through centre of the cylinder. The cylinder has a length I rotating about its axis, where the length is sufficiently long that the area (πR²) of an end portion is small compared to an area of the curved sides (2πRl) and where R<<δ_{μG}, and R=R_{G}, the damping factor, C_{F}, stiffness loading factor, K_{F}, and inertial loading factor, J_{F}, may be written as:

$${C}_{F} = A R {μ}^{′} = ( 2 π {R}^{2} l ) {μ}^{′} \qquad ( Equation 27 )$$

$${K}_{F} = A R {G}^{′} = ( 2 π {R}^{2} l ) {G}^{′} \qquad ( Equation 28 )$$

$${J}_{F} = A {R}^{3} ρ = ( 2 π {R}^{4} l ) ρ . \qquad ( Equation 29 )$$

While geometric damping through the propagation of radial waves brings advantages through a dependency on geometric parameters rather than fluid properties, the conditions for geometric damping encourage the use of a cylindrical element with a small radius, which results in a small active surface area. The small R² term in the damping, stiffness and inertial loading factors means that the damping, or elastic or inertial loading factors from torsional vibration are small, even at high viscosities.

FIG. 3 also shows a second cylinder 30 having the same size as the first cylinder 20, wherein the second cylinder 30 is displaced perpendicularly from the axis of the axis 25 of the first cylinder 20 by an offset radius R_{O} that is greater than R. The second cylinder 30 is also vibrated torsionally about the axis 25 of the first cylinder 20. This has the effect of changing the radius of gyration R_{G} from R (the distance from the cylindrical surface to the axis 25) to R_{O} (the radial offset of the cylinder as a whole).

If the equations provided above for the damping factor, C_{F}, stiffness loading factor, K_{F}, and inertial loading factor, J_{F}, under geometric damping hold (i.e. negligible non-geometric damping), then the equations may be expressed as:

$${C}_{F} = ( A \cdot \frac{{R}_{O}^{2}}{R} ) {μ}^{′} = ( \frac{2 π R l {R}_{O}^{2}}{R} ) {μ}^{′} = ( 2 π l {R}_{O}^{2} ) {μ}^{′} \qquad ( Equation 30 )$$

$${K}_{F} = ( A \cdot \frac{{R}_{O}^{2}}{R} ) {G}^{′} = ( \frac{2 π R l {R}_{O}^{2}}{R} ) {G}^{l} = ( 2 π l {R}_{O}^{2} ) {G}^{′} \qquad ( Equation 31 )$$

$${J}_{F} = ( A \cdot {R}_{O}^{2} \cdot R ) ρ = ( 2 π R l {R}_{O}^{2} R ) ρ = ( 2 π l {R}_{O}^{4} ) ρ . \qquad ( Equation 32 )$$

If Ro is greater than R then, by offsetting the vibration of the cylindrical element from the axis, the loading factors are amplified the square of the ratio between R_{O} and R, i.e. an amplification of (R_{O}/R) 2. The loading factors are amplified by (R_{O}/R) 4 in the case of J_{F}, i.e. the fourth power of the ratio between R_{O} and R.

However, these equations only hold under geometric damping of the cylindrical element. By offsetting the cylindrical element, it no longer undergoes pure torsional oscillation but instead vibrates laterally at the displaced distance. These loading factor equations do not automatically apply because, under lateral vibration, the cylindrical element may form a dipole wave field rather than a monopole wave field.

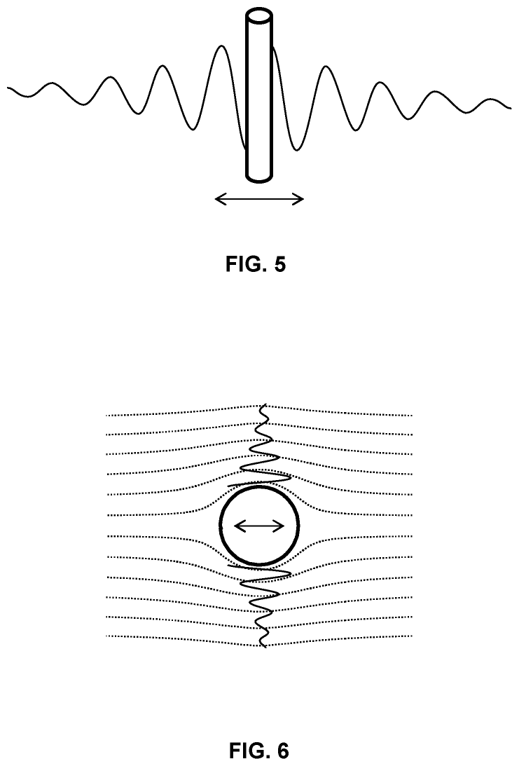

FIG. 5 illustrates a cylindrical element under lateral vibration causing a dipole wave field. Under a dipole wave field, there is a 180° phase difference between the wave field on each side of the cylindrical element. The formation of a dipole wave field is problematic because the resulting waves are pressure (P) waves rather than shear(S) waves. The fluid mechanics of pressure waves are different from shear waves and the previously defined relationships for shear waves no longer apply.

For example, a damping factor is defined differently for a shear wave than for a pressure wave. For a shear wave, the shear rate and the stresses created by the shear rate lead to a relatively well-defined and controllable damping wave. By contrast, pressure waves follow what is known as ‘quadratic damping’, where the damping force is proportional to the square of the velocity. This leads to a damping factor of the form:

$${C}_{quadratic} = b \cdot v \qquad ( Equation 33 )$$

where b is a constant and v is the velocity. In other words, the damping factor unhelpfully varies with the vibrational velocity.

However, a monopole wave field may be maintained if the Reynolds number is kept low. The Reynolds number signifies a ratio between inertial forces and viscous forces. As the Reynolds number reduces, viscous forces become larger relative to inertial forces.

FIG. 6 illustrates laminar flow around a cylinder vibrating in a left-right direction perpendicular to the cylinder's axis. With laminar flow, the forces across either side of the cylinder become shear and so shear waves propagate as a result of lateral vibration. Without wishing to be bound by theory, it is believed that laminar flow leads to inertial forces being sufficiently low relative to viscous forces that the wave field is largely or at least partially defined by shear waves generated from the upper and lower portions of the cylinder cross-section, i.e. perpendicular to the axis and the direction of vibration. As a result, as the Reynolds number is reduced, the degree to which the wave field has a dipole form is reduced and the degree to which the wave field has a monopole form is increased. The low Reynolds number has restored a partial shear wave field which is in phase through out its propagation space. The benefits of geometric damping are retained but with the advantage of increased loading factor gain from offsetting the element from the axis.

Advantageously, the cylindrical element displaced from the axis may achieve a relatively high degree of amplification with little increase in size and weight because a relatively low Reynolds number is readily achievable with small length scales. In some implementations of the techniques of this disclosure, fluid loading factor may be achieved that are equivalent to those of much larger and heavier vibratory elements.

It is further recognised that a relatively low Reynolds number is readily achievable for micro- and nanoscale devices for almost all fluids of interest, regardless of their physical properties. Sub-micron needle structures on a vibrating substrate may form the same radially displaced elements discussed above and achieve the same benefits of geometric damping. Some implementations may feature multiple cylindrical elements or cylinder-like elements, such as pins and spikes, formed by micro- or nano-manufacturing processes, may allow miniature surfaces to present high fluid load factors.

It is further recognised that a low Reynolds number may lead to a wave field is that is only partially defined by shear waves and so expressions for the loading factor under geometric damping conditions might not be identical to the expressions set out above, but may be proportional to those expressions, the constant of proportionality depending on the degree to which the wave field is defined by shear waves. In the following expressions, a constant of proportionality, h, is introduced, wherein h would have a value of 1 if the wave field is wholly defined by shear waves, and would have a value of 0.5 if the wave field is 50% defined by shear waves, which may be a reasonable assumption in practice:

$${C}_{F} = h ( 2 π l {R}_{O}^{2} ) {μ}^{′} \qquad ( Equation 34 )$$

$${K}_{F} = h ( 2 π l {R}_{O}^{2} ) {G}^{′} \qquad ( Equation 35 )$$

$${J}_{F} = h ( 2 π l {R}_{O}^{4} ) ρ . \qquad ( Equation 36 )$$

If h is assumed to be 0.5, then the above expressions simplify to:

$${C}_{F} = ( π {lR}_{O}^{2} ) {μ}^{′} \qquad ( Equation 37 )$$

$${K}_{F} = ( π {lR}_{O}^{2} ) {G}^{′} \qquad ( Equation 38 )$$

$${J}_{F} = ( π {lR}_{O}^{4} ) ρ . \qquad ( Equation 39 )$$

It is further recognised that the techniques of this disclosure may have particular application in the measurement of properties of yield stress fluids following techniques described in WO 2018/197902 A1 of Hydramotion Ltd (‘The Measurement Of Properties Of Flowing Yield Stress Fluids’) and WO 2018/197900 A1 of Hydramotion Ltd (‘The Measurement Of Properties Of Vibrated Yield Stress Fluids’), the entire contents of both documents being hereby incorporated by reference into the present disclosure.

The meaning of a ‘low’ Reynolds number in the context of the present disclosure is that the Reynolds number is sufficiently low that laminar flow is obtained and the flow due to the vibration may be characterised to some degree by a shear wave field and the advantages of geometric damping provided to at least some degree. It is recognised that a laminar-to-turbulent flow transition occurs over a range of Reynolds number values and the precise range over which this transition takes place is dependent on geometry. A lower Reynolds number is more likely to result in flow behaviour leading to a partial shear wave field than a higher Reynolds number. Without wishing to be bound by theory, it is believed that the degree to which a shear wave field develops, and thus some advantages of the techniques of this disclosure are obtained, is dependent on the Reynolds number. For example, a Reynolds number of 1000 may exhibit a degree of laminar-like flow and result in a shear wave field to some degree. A Reynolds number of 100 may exhibit an even greater degree of laminar-like flow and result in a shear wave field to an even greater degree. A Reynolds number of 10 may exhibit an even greater degree of laminar-like flow and result in a shear wave field to an even greater degree. A Reynolds number of 1 may exhibit an even greater degree of laminar-like flow and result in a shear wave field to an even greater degree. In general, a lower Reynolds number may be preferred but the reader will recognise that achieving a lowest possible Reynolds number has to be balanced with other technical considerations.

In these disclosures, a description of a yield stress fluid's viscoplastic boundary region is presented along with techniques to determine physical properties of fluids by transmitting shear waves of varying propagation depths, some extending only into viscoplastic boundary region and some extending through the liquid boundary region. The techniques of this disclosure allow that, provided geometric damping conditions are satisfied, the wave propagation depth is determined by the geometry of the cylindrical element, and specifically its radius. By using multiple cylindrical elements including at least two of differing radius, the cylindrical elements will generate waves that propagate known distances into the yield stress fluid, allowing the straightforward design and specification of measurement systems to probe the liquid boundary layer and beyond of a yield stress fluid such as a flowing yield stress fluid.

Such techniques may comprise a method of estimating the yield stress of a flowing yield stress fluid using one or more vibratory transducers having a vibratory surface in contact with the yield stress fluid, the method comprising: vibrating a vibratory surface of a vibratory transducer to transmit a wave from a vibrating surface into a viscoplastic boundary layer of the flowing yield stress fluid; making, using the vibrations of the vibratory transducer, one or more measurements of the degree of damping of vibration; and estimating the yield stress of the flowing yield stress fluid based on the one or more measurements of the degree of damping of vibration, wherein the one or more vibratory transducers include multiple cylindrical elements as described herein, optionally offset from an axis a vibratory transducer, whether on a single vibratory transducer or distributed over multiple vibratory transducers, wherein a criterion for geometric damping is satisfied.

For example, a first measurement of the degree of damping of vibration may be made with the vibratory surface of a vibratory transducer vibrating at a first frequency of vibration to transmit a wave that propagates a first distance into the viscoplastic boundary layer of the flowing yield stress fluid, and a second measurement of the degree of damping of vibration is made with the vibratory surface of a vibratory transducer vibrating at a second frequency of vibration that is different from the first frequency to transmit a wave that propagates a second distance into the viscoplastic boundary layer of the flowing yield stress fluid that is lower than the first distance, and the yield stress of the flowing yield stress fluid may be estimated based on a linear combination of the first and second measurements of the degree of damping of vibration. The method may further comprise performing a correction to one or both of the first and second measurements of the degree of damping of vibration based on the first and second frequencies of vibration and the power law index of the yield stress fluid. The estimate of the yield stress of the flowing yield stress fluid is proportional to:

$$( V 1 - V 2 {( \frac{ω 1}{ω 2} )}^{n - 1} )$$

wherein V1 is the first measurement of the degree of damping of vibration, V2 is the second measurement of the degree of damping of vibration, ω1 is the angular frequency of the first frequency of vibration, ω2 is the angular frequency of the second frequency of vibration, and n is the power law index. The method may further comprise: i) making a third measurement of the degree of damping of vibration with the vibratory surface of a vibratory transducer vibrating at a third frequency of vibration that is different from the first and second frequencies of vibration to transmit a wave that propagates a third distance into the viscoplastic boundary layer of the flowing yield stress fluid that is less than the first distance; and ii) estimating the power law index of the flowing yield stress fluid based on the third measurement of the degree of damping of vibration and the third frequency of vibration and further based on one of: the first measurement of the degree of damping of vibration and the first frequency of vibration; and the second measurement of the degree of damping of vibration and the second frequency of vibration. Thus the generation of shear waves of known propagation depths by way of the techniques of this disclosure is readily and advantageously applied.

The techniques of this disclosure also permit the determining of whether a cylindrical element is radiating waves in a monopole-like pattern or a dipole-like pattern and permit making inferences based on such a determination.

The damping of a dipole does not follow shear wave theory as the dipole produces P waves, under which a magnitude of a damping force is given by av², where a is a constant and v is the fluid velocity. In other words, the damping force is quadratic with velocity. This results in a damping factor given by (Equation 33). In other words, the damping factor varies with velocity.

However, the geometric damping described herein is independent of vibrational velocity, as set out above in connection with (Equation 30), (Equation 31) and (Equation 32) for example.

The vibrational velocity is a function of the vibrational amplitude, and varying the vibrational amplitude will vary the vibrational velocity. By altering the vibrational amplitude and making measurements of vibration within a fluid at different vibrational amplitudes (i.e. without changing frequency), the degree of dipole behaviour of the wave field around the cylindrical element, or ‘dipolarity’, can be detected. The dipolarity can be considered a quantity indicative of the degree to which the wave field has the form of a dipole rather than a monopole. In particular, a change in Q factor will indicate a change in damping factor because the Q factor is inversely proportional to the damping factor.

Therefore a change in Q factor is proportional to the degree of dipolarity, i.e.:

$$Δ Q ∝ Dipolarity \qquad ( Equation 40 )$$

In addition, the dipolarity is a function of Reynolds number, given by Re=2Rvρ/μ′. Therefore:

$$Δ Q ∝ Re = 2 Rv ρ / {μ}^{′} . \qquad ( Equation 41 )$$

The variation of Q by modulating velocity (in turn by modulating amplitude of vibration) can therefore be used to indicate a change in Reynolds number, which is useful in determining whether the monopole-like behaviour of geometric damping is present, or as an alternative means for determining parameters of the Reynolds number, such as velocity, viscosity or density.

A change from a vibrating element in a fluid causing a wave field with high dipolarity, in which the generated waves are acoustic (i.e. pressure) waves, to causing a wave field of a monopole or near monopole, in which the generated waves are only or mostly shear waves requires that the disturbed fluid around the vibrating element is laminar as the element vibrates and moves through the fluid. The condition for laminar flow is therefore a low Reynolds number in the small-amplitude vibration condition, such as a Reynolds number less than 1000, less than 100, less than 10, or less than 1, where a lower Reynolds number indicates a greater degree of laminar flow.

The determining of parameters of the Reynolds number such as viscosity in particular may be advantageous because the length scale and density may be assumed to be fixed in a system. The variation in Reynolds number may be governed wholly or mostly by the ratio between velocity and viscosity. At very high viscosity, the Reynolds number will be small even for a wide range of vibrational velocities. At low viscosity, the variation in velocity can modulate the Reynolds number to a higher value, causing the loss of the laminar flow condition and the generation of acoustic waves. The acoustic waves follow quadratic damping, which varies with the square of velocity, and so a variation in amplitude of vibration changes the local velocity leading to a detectable damping change. Thus an estimate of the Reynolds number may be obtained from the difference in Q factors.

In some examples according to the techniques of this disclosure, a change in Reynolds number may be considered to be proportional to the ratio between the change in Q factor and the change in amplitude of vibration:

$$Δ Q / Δ A ∝ Δ Re , \qquad ( Equation 42 )$$

where ΔA represents a change in amplitude and ΔRe represents a change in Reynolds number. If ΔQ/ΔA is zero or negligible, then the Reynolds number is low and the vibrational flow may be estimated to be laminar. If ΔQ/ΔA is non-zero or greater than a threshold value, then the fluid flow is not completely laminar and the Reynolds number has increased. The degree to which the Reynolds number has increased is dependent on the value of ΔQ/ΔA.

In the case of determining a Reynolds number or determining whether the monopole-like behaviour of geometric damping is present (or the degree to which it is present), this technique represents determining a property of the fluid wherein the determined property of the fluid is a property of the flow of the fluid due to the vibration of the vibratory transducer element in the fluid at the frequency of vibration for one or both of the first and second amplitudes. Determining a velocity, viscosity or density based on a determined Reynolds number also represents determining a property of the fluid that is a property of the flow of the fluid due to the vibration of the vibratory transducer element in the fluid at the frequency of vibration for one or both of the first and second amplitudes.

These techniques making use of the change in Q factor between vibration at different amplitudes to determine fluid properties such as properties of the flow of the fluid around the vibrating element or properties of the fluid itself such as viscosity may be applied in a fluid with a yield stress. These techniques may also be applied in a fluid with zero yield stress because they do not depend on the fluid having a yield stress.

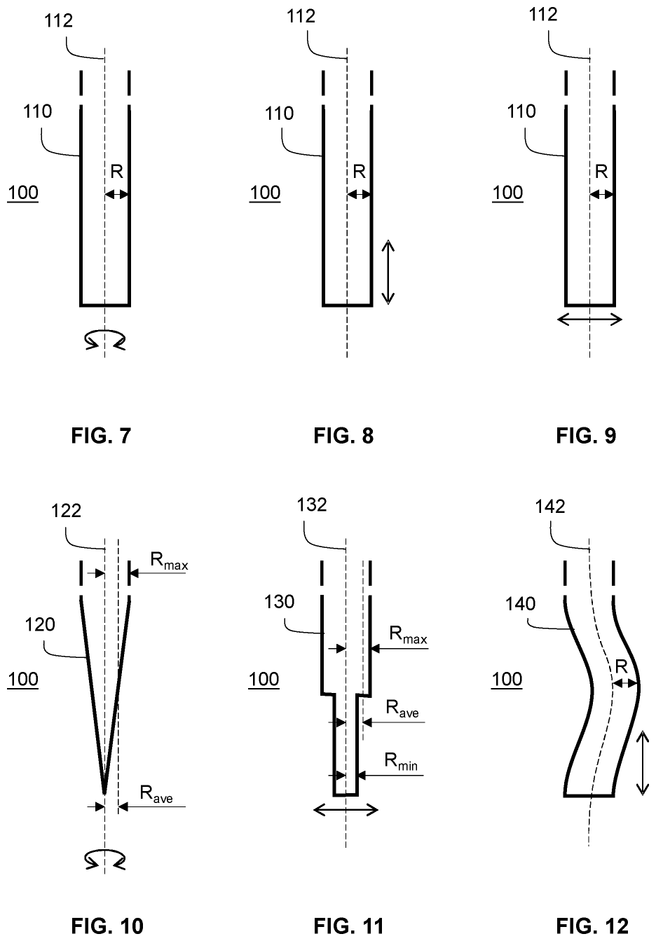

FIGS. 7 to 31 illustrate vibratory transducers or portions thereof in accordance with the techniques of this disclosure or for use with processes in accordance with the techniques of this disclosure. While these drawings illustrate vibration relative to axes in particular directions, such as torsionally, laterally or longitudinally, it will be recognised that the techniques of this disclosure are not limited to any particular direction of vibration. Vibratory transducers with vibrating elements in accordance with the techniques of this disclosure may be configured to vibrate in multiple directions of vibrations, including combinations of torsional and lateral vibration, or torsional and longitudinal vibration. Moreover, the foregoing discussion has treated the vibrating element as a cylinder. However, while this may simplify the analysis, the vibrating element is not required to be a cylinder or even have a perfectly circular cross section to achieve at least some of the advantages identified herein. Thus at least some of the vibratory transducers or portions thereof are discussed using the more general expression ‘elongate member’.

FIGS. 7 to 9 each illustrate a vibratory transducer for use with the techniques of this disclosure. In each of FIGS. 7 to 9, the vibratory transducer has a cylindrical vibrating element 110 in contact with a fluid 100. The cylindrical vibrating element has a radius R about an axis 112 along the length of the cylindrical vibrating element. In accordance with the techniques of this disclosure, geometric damping is obtained when the radius R is less than the viscoelastic propagation depth of a shear wave in the fluid 100, which is a function of the angular frequency of vibration, ω and physical properties of the fluid 100.

In FIG. 7, the cylindrical vibrating element 110 is configured to vibrate torsionally about the axis 112 with an angular frequency, ω.

In FIG. 8, the cylindrical vibrating element 110 is configured to vibrate longitudinally along the axis 112 with an angular frequency, ω.

In FIG. 9, the cylindrical vibrating element 110 is configured to vibrate laterally in a direction perpendicular to the axis 112 with an angular frequency, ω.

FIGS. 10 and 11 illustrate vibratory transducers for use with the techniques of this disclosure. In each of FIGS. 10 and 11, a vibratory transducer has an elongate member in contact with a fluid, the elongate member having a circular cross section along the length of the long axis through the elongate member and the elongate member is configured to vibrate torsionally about the long axis with an angular frequency, ω.

In FIG. 10, at an end portion of the elongate member 120, the radius of the circular cross section decreases linearly with position along axis 122, one end of the elongate member 120 forming a conical shape.

In FIG. 11, the radius of the circular cross section along the elongate member 130 decreases with position along axis 132 discontinuously in a piecewise constant fashion, the radius stepping from a maximum radius Rₘₐₓ to a minimum radius Rₘᵢₙ, the elongate member 130 having an average radius Rₐᵥₑ along the length of the axis 132.

Geometric damping will take place along at least a portion of the elongate member if a portion of the elongate member has a radius that is less than the viscoelastic propagation depth in the fluid 100. In accordance with the techniques of this disclosure, a beneficial level of geometric damping is obtained when an average radius, Rₐᵥₑ, of the elongate member along the length of the axis is less than a viscoelastic propagation depth of a shear wave in the fluid 100. A higher level of geometric damping may be obtained if a maximum radius, Rₘₐₓ, is less than the viscoelastic propagation depth.

FIG. 12 illustrates a vibratory transducer for use with the techniques of this disclosure. The vibratory transducer has an elongate member 140 in contact with a fluid 100. The elongate member 140 is not straight but instead has a curve. An axis 142 extends lengthwise through the centre of the elongate member. The axis 142 is itself curved. The elongate member is configured to vibrate in a linear direction that is broadly aligned with the axis 142. The elongate member 140 has a circular cross section along its length despite the curvature and has a radius R about the axis 142. In accordance with the techniques of this disclosure, geometric damping is obtained when the radius R is less than the viscoelastic propagation depth of a shear wave in the fluid 100, which is a function of the angular frequency of vibration, ω and physical properties of the fluid 100.

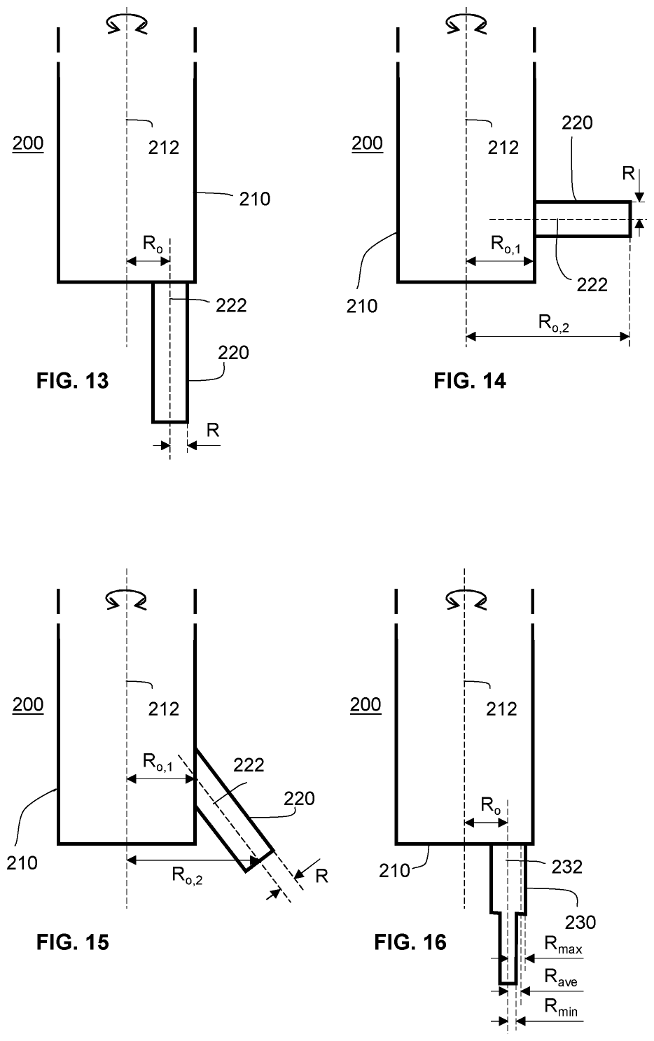

FIGS. 13 to 20 illustrate further vibratory transducers for use with the techniques of this disclosure in which elongate members are connected to shafts that have longitudinal axes, the elongate members offset from the longitudinal axes of the shafts.

FIG. 13 illustrates a vibratory transducer for use with the techniques of this disclosure. The vibratory transducer comprises a shaft 210 and an elongate member 220 attached to the shaft. The shaft 210 is cylindrical and is configured to vibrate torsionally about a longitudinal axis 212 through the centre of the shaft 210 at an angular frequency of vibration, ω. The elongate member 220 is also cylindrical and is connected to the shaft 210 at one end of the shaft 210. The elongate member 220 has a longitudinal axis 222 that is parallel to the axis 212 of the shaft 210 about which the shaft 210 vibrates but radially offset from the axis 212 of the shaft 210 by an offset distance R_{O}. The elongate member 220 has a radius R. The shaft 210 and the elongate member 220 are in contact with a fluid 200. In accordance with the techniques of this disclosure, a beneficial level of geometric damping is obtained when the viscoelastic propagation distance of a shear wave in the fluid 200 at the angular frequency of vibration is greater than the radius R of the elongate member 220 and when a Reynolds number of flow of the fluid 200 around the elongate member 220 is low. The offsetting of the elongate member 220 from the axis 212 of the shaft 210 provides increased damping than would have been obtained if the axis 222 of the elongate member 220 was in line with the axis 212 of the shaft 210.

FIG. 14 illustrates a vibratory transducer for use with the techniques of this disclosure. The vibratory transducer comprises a shaft 210 and an elongate member 220 attached to the shaft. The shaft 210 is cylindrical and is configured to vibrate torsionally about a longitudinal axis 212 through the centre of the shaft 210 at an angular frequency of vibration, ω. The elongate member 220 is also cylindrical and is connected to the shaft 210. In contrast with the arrangement illustrated in FIG. 13, the elongate member 220 of FIG. 14 is connected to the curved side wall of the shaft 210 and has a longitudinal axis 222 that is perpendicular to the axis 212 of the shaft 210 about which the shaft 210 vibrates. The elongate member 220 has a radius R. A proximal end of the elongate member 220 is offset from the axis 212 of the shaft 210 by a first offset distance R_{O,1} that is equal to a radius of the shaft 210. A distal end of the elongate member 220 is offset from the axis 212 of the shaft 210 by a second offset distance R_{O,2} that is equal to the sum of the radius of the shaft 210 and the length of the elongate member 220. The elongate member 220 is offset from the axis 212 of the shaft 210 along its entire length. The shaft 210 and the elongate member 220 are in contact with a fluid 200. In accordance with the techniques of this disclosure, a beneficial level of geometric damping is obtained when the viscoelastic propagation distance of a shear wave in the fluid 200 at the angular frequency of vibration is greater than the radius R of the elongate member 220 and when a Reynolds number of flow of the fluid 200 around the elongate member 220 is low. The offsetting of the elongate member 220 from the axis 212 of the shaft 210 provides increased damping than would have been obtained if the axis 222 of the elongate member 220 was in line with the axis 212 of the shaft 210.

FIG. 15 illustrates a vibratory transducer for use with the techniques of this disclosure. The vibratory transducer comprises a shaft 210 and an elongate member 220 attached to the shaft. The shaft 210 is cylindrical and is configured to vibrate torsionally about a longitudinal axis 212 through the centre of the shaft 210 at an angular frequency of vibration, ω. The elongate member 220 is also cylindrical and is connected to the shaft 210. In contrast with the arrangement illustrated in FIG. 13, the elongate member 220 of FIG. 15 is connected to the curved side wall of the shaft 210. In contrast with the arrangements illustrated in FIG. 13 and FIG. 14, the elongate member 220 of FIG. 15 extends in an oblique direction that is neither wholly radial nor wholly axial. The elongate member 220 has a longitudinal axis 222 that is oblique to the axis 212 of the shaft 210 about which the shaft 210 vibrates. The elongate member 220 has a radius R. A proximal end of the elongate member 220 is offset from the axis 212 of the shaft 210 by a first offset distance R_{O,1} that is equal to a radius of the shaft 210. A distal end of the elongate member 220 is offset from the axis 212 of the shaft 210 by a second offset distance R_{O,2} that is equal to the sum of the radius of the shaft 210 and a radial component of the length of the elongate member 220. The elongate member 220 is offset from the axis 212 of the shaft 210 along its entire length. The shaft 210 and the elongate member 220 are in contact with a fluid 200. In accordance with the techniques of this disclosure, a beneficial level of geometric damping is obtained when the viscoelastic propagation distance of a shear wave in the fluid 200 at the angular frequency of vibration is greater than the radius R of the elongate member 220 and when a Reynolds number of flow of the fluid 200 around the elongate member 220 is low. The offsetting of the elongate member 220 from the axis 212 of the shaft 210 provides increased damping than would have been obtained if the axis 222 of the elongate member 220 was in line with the axis 212 of the shaft 210.

FIG. 16 illustrates a vibratory transducer for use with the techniques of this disclosure. The vibratory transducer comprises a shaft 210 and an elongate member 230 attached to the shaft. The shaft 210 is cylindrical and is configured to vibrate torsionally about a longitudinal axis 212 through the centre of the shaft 210 at an angular frequency of vibration, ω. The elongate member 230 is connected to the shaft 210 at one end of the shaft 210 and has a longitudinal axis 232 that extends in a direction parallel to the axis 212 of the shaft 210 but is offset from the axis 212 of the shaft by an offset distance R_{O}. In contrast with the arrangement illustrated in FIG. 13, while the elongate member 230 has a circular cross section along its length, the elongate member 230 does not have a constant radius along its length. Instead, the elongate member 230 comprises a first cylindrical portion having a first radius and a second cylindrical portion having a second radius that is different to the first radius, the first cylindrical portion connected to the shaft 210 and the second cylindrical portion connected to the first cylindrical portion. The first and second cylindrical portions of the elongate member 230 share the same common axis 232. The first radius is a maximum radius Rₘₐₓ of the elongate member 230 and the second radius is a minimum radius Rₘᵢₙ of the elongate member 230. The average radius of the elongate member 230 along its length is Rₐᵥₑ. The shaft 210 and the elongate member 230 are in contact with a fluid 200. In accordance with the techniques of this disclosure, a beneficial level of geometric damping is obtained when the viscoelastic propagation distance of a shear wave in the fluid 200 at the angular frequency of vibration is greater than Rₘᵢₙ (more preferably greater than Rₐᵥₑ, more preferably greater than Rₘₐₓ) and when a Reynolds number of flow of the fluid 200 around the elongate member 230 is low. The offsetting of the elongate member 230 from the axis 212 of the shaft 210 provides increased damping than would have been obtained if the axis 232 of the elongate member 230 was in line with the axis 212 of the shaft 210.

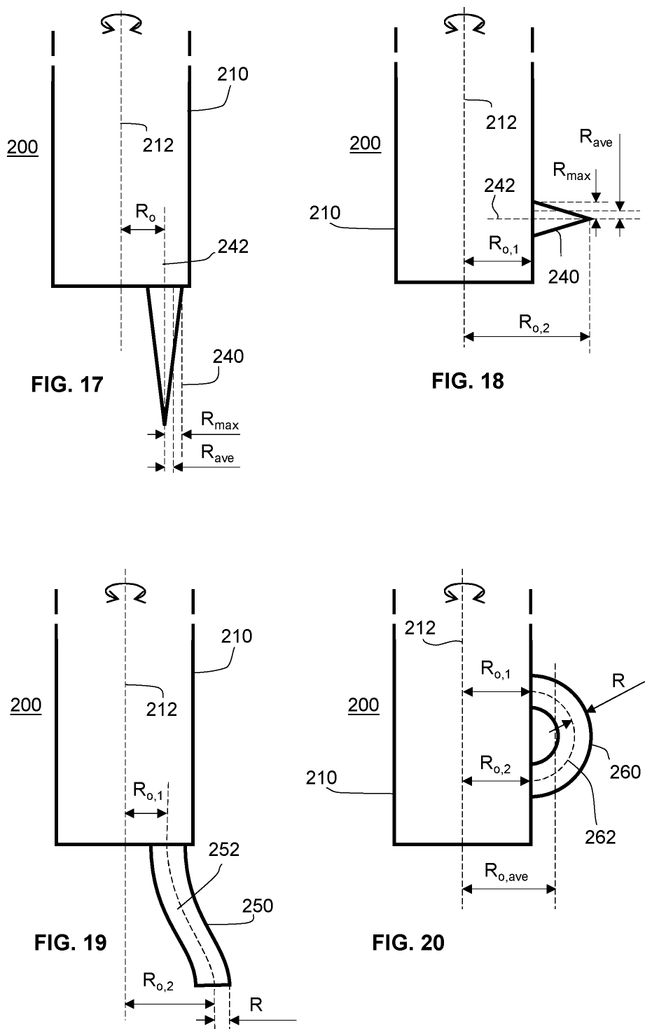

FIG. 17 illustrates a vibratory transducer for use with the techniques of this disclosure. The vibratory transducer comprises a shaft 210 and an elongate member 240 attached to the shaft. The shaft 210 is cylindrical and is configured to vibrate torsionally about a longitudinal axis 212 through the centre of the shaft 210 at an angular frequency of vibration, ω. The elongate member 240 is connected to the shaft 210 at one end of the shaft 210 and has a longitudinal axis 242 that extends in a direction parallel to the axis 212 of the shaft 210 but is offset from the axis 212 of the shaft by an offset distance R_{O}. In contrast with the arrangement illustrated in FIG. 13, while the elongate member 240 has a circular cross section along its length, the elongate member 240 does not have a constant radius along its length. Instead, the elongate member 240 is conical, having a maximum radius Rₘₐₓ where the elongate member 240 is connected to the shaft 210. The average radius of the elongate member 240 along its length is Rₐᵥₑ. The shaft 210 and the elongate member 240 are in contact with a fluid 200. In accordance with the techniques of this disclosure, a beneficial level of geometric damping is obtained when the viscoelastic propagation distance of a shear wave in the fluid 200 at the angular frequency of vibration is greater than Rₐᵥₑ (more preferably greater than Rₘₐₓ) and when a Reynolds number of flow of the fluid 200 around the elongate member 240 is low. The offsetting of the elongate member 240 from the axis 212 of the shaft 210 provides increased damping than would have been obtained if the axis 242 of the elongate member 240 was in line with the axis 212 of the shaft 210.

FIG. 18 illustrates a vibratory transducer for use with the techniques of this disclosure. The vibratory transducer comprises a shaft 210 and an elongate member 240 attached to the shaft. The shaft 210 is cylindrical and is configured to vibrate torsionally about a longitudinal axis 212 through the centre of the shaft 210 at an angular frequency of vibration, ω. The elongate member 240 is connected to the shaft 210. The elongate member 240 is conical, having a maximum radius Rₘₐₓ where the elongate member 240 is connected to the shaft 210. The average radius of the elongate member 240 along its length is Rₐᵥₑ. In contrast with the arrangement illustrated in FIG. 17, the elongate member 240 of FIG. 18 is connected to the curved side wall of the shaft 210 and has a longitudinal axis 242 that is perpendicular to the axis 212 of the shaft 210 about which the shaft 210 vibrates. A proximal end of the elongate member 240 is offset from the axis 212 by a first offset distance R_{O,1} that is equal to a radius of the shaft 210. A distal end of the elongate member 240 is offset from the axis 212 by a second offset distance R_{O,2} that is equal to the sum of the radius of the shaft 210 and the length of the elongate member 240. The whole of the elongate member 240 (including both ends) is offset from the axis 212 of the shaft 210. The shaft 210 and the elongate member 240 are in contact with a fluid 200. In accordance with the techniques of this disclosure, a beneficial level of geometric damping is obtained when the viscoelastic propagation distance of a shear wave in the fluid 200 at the angular frequency of vibration is greater than Rₐᵥₑ (more preferably greater than Rₘₐₓ) and when a Reynolds number of flow of the fluid 200 around the elongate member 240 is low. The offsetting of the elongate member 240 from the axis 212 of the shaft 210 provides increased damping than would have been obtained if the axis 242 of the elongate member 240 was in line with the axis 212 of the shaft 210.

FIG. 19 illustrates a vibratory transducer for use with the techniques of this disclosure. The vibratory transducer comprises a shaft 210 and an elongate member 250 attached to the shaft. The shaft 210 is cylindrical and is configured to vibrate torsionally about a longitudinal axis 212 through the centre of the shaft 210 at an angular frequency of vibration, ω. The elongate member 250 is connected to the shaft 210. The elongate member 250 is not straight but instead has a curve. An axis 252 extends lengthwise through the centre of the elongate member 250. The axis 252 is itself curved. The elongate member 250 has a circular cross section along its length despite the curvature and has a radius R about the axis 242. The elongate member 250 is connected to the shaft 210 at an end of the shaft, extending in a direction that is broadly in line with the axis 210 of the shaft, but the elongate member 250 is offset from the axis 212 of the shaft 210. A proximal end of the elongate member 250 is offset from the axis 212 of the shaft 210 by a first offset distance R_{O,1} and a distal end of the elongate member 250 is offset from the axis 212 of the shaft 210 by a second distance R_{O,2}. The axis 252 extending lengthwise through the centre of the elongate member 250 is offset from the axis 212 of the shaft 210 along the whole length of the elongate member 250. The shaft 210 and the elongate member 250 are in contact with a fluid 200. In accordance with the techniques of this disclosure, a beneficial level of geometric damping is obtained when the viscoelastic propagation distance of a shear wave in the fluid 200 at the angular frequency of vibration is greater than R and when a Reynolds number of flow of the fluid 200 around the elongate member 250 is low. The offsetting of the elongate member 250 from the axis 212 of the shaft 210 provides increased damping than would have been obtained if the axis 252 of the elongate member 250 was in line with the axis 212 of the shaft 210.

FIG. 20 illustrates a vibratory transducer for use with the techniques of this disclosure. The vibratory transducer comprises a shaft 210 and an elongate member 260 attached to the shaft. The shaft 210 is cylindrical and is configured to vibrate torsionally about a longitudinal axis 212 through the centre of the shaft 210 at an angular frequency of vibration, ω. The elongate member 260 is not straight but instead has a curve. The elongate member 260 has a first end and a second end. Both first and second ends of the elongate member 260 are connected to the shaft 210 at locations spaced longitudinally (in a direction aligned with the axis 212 of the shaft) from each other along the curved side wall of the shaft 210, the elongate member 260 forming a closed loop with the shaft 210. An axis 262 extends lengthwise through the centre of the elongate member 260 along a curved path. The elongate member 260 has a circular cross section along its length despite the curvature and has a radius R about the axis 262. In FIG. 20, the elongate member 260 has the form of a semi-torus. Both first and second ends of the elongate member 260 are offset from the axis 212 of the shaft 210 by first and second offset distances, R_{O,1} and R_{O,2}, that are both equal to the radius of the shaft 210. The axis 262 of the elongate member 260 is offset from the axis 210 of the shaft 210 along the whole length of the elongate member 260. The average offset distance R_{O,ave} of the elongate member 262 along the length of the elongate member 260 is greater than the offset distances R_{O,1} and R_{O,2} at either end of the elongate member 260. The shaft 210 and the elongate member 250 are in contact with a fluid 200. In accordance with the techniques of this disclosure, a beneficial level of geometric damping is obtained when the viscoelastic propagation distance of a shear wave in the fluid 200 at the angular frequency of vibration is greater than R and when a Reynolds number of flow of the fluid 200 around the elongate member 260 is low. The offsetting of the elongate member 260 from the axis 212 of the shaft 210 provides increased damping than would have been obtained if the axis 262 of the elongate member 260 was in line with the axis 212 of the shaft 210.

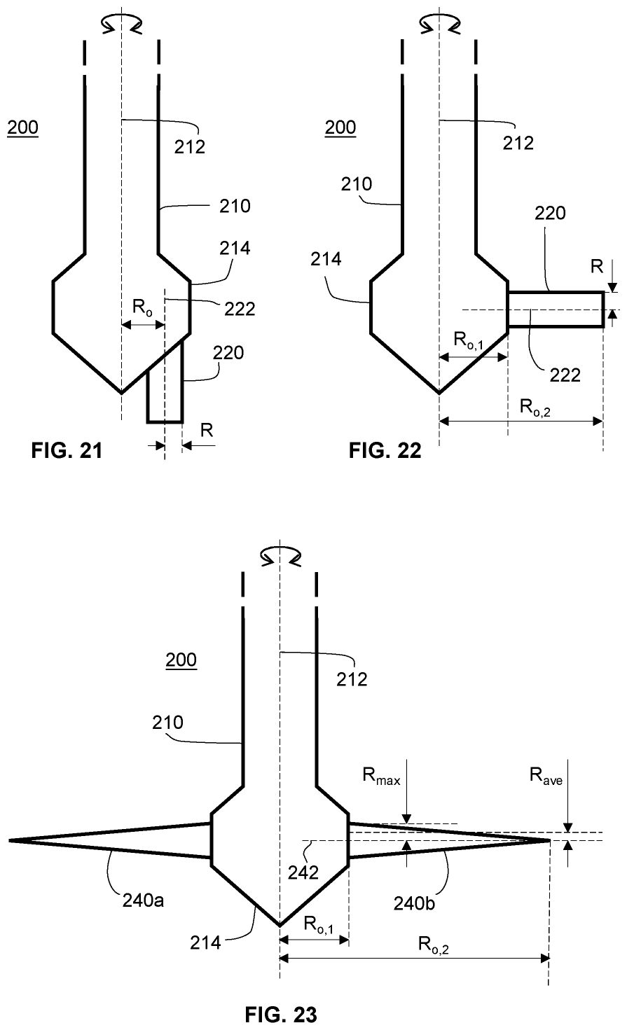

FIGS. 21 to 23 illustrate vibratory transducers for use with the techniques of this disclosure that are different from the vibratory transducers illustrated in FIGS. 13 to 20. While the vibratory transducers illustrated in FIGS. 21 to 23 include elongate members connected to shafts, they differ in that each shaft includes a bob at an end of the shaft, the elongate members connected to the shaft at the bob.

FIG. 21 illustrates a vibratory transducer for use with the techniques of this disclosure. The vibratory transducer comprises a shaft 210 and a bob 214 at an end of the shaft 210 and an elongate member 220 at connected to the shaft 210 at the bob 214. The shaft 210 is cylindrical and is configured to vibrate torsionally about a longitudinal axis 212 through the centre of the shaft 210 at an angular frequency of vibration, ω. The bob 214 represents an end portion of the shaft 210 that has a larger diameter than the rest of the shaft 210. The bob 214 has three axial sections that are axially aligned with the axis 212 of the shaft 210, including: i) a first axial section that is frustoconical, where the radius increases linearly across the axial section from an initial radius, that is equal to the shaft radius, to a final radius; ii) a second axial section that is cylindrical and has a radius equal to the final radius of the first axial section; and iii) a third axial section that is conical, where the radius decreases linearly from the radius of the second axial section to zero. The elongate member 220 is cylindrical and has a radius R and is connected to the third axial section of the bob 214. The elongate member 220 has a longitudinal axis 222 that is parallel to the axis 212 of the shaft 210 about which the shaft 210 vibrates but radially offset from the axis 212 of the shaft 210 by an offset distance R_{O}. The shaft 210 (including the bob 214) and the elongate member 220 are in contact with a fluid 200. In accordance with the techniques of this disclosure, a beneficial level of geometric damping is obtained when the viscoelastic propagation distance of a shear wave in the fluid 200 at the angular frequency of vibration is greater than the radius R of the elongate member 220 and when a Reynolds number of flow of the fluid 200 around the elongate member 220 is low. The offsetting of the elongate member 220 from the axis 212 of the shaft 210 provides increased damping than would have been obtained if the axis 222 of the elongate member 220 was in line with the axis 212 of the shaft 210.

FIG. 22 illustrates a vibratory transducer for use with the techniques of this disclosure. The vibratory transducer comprises a shaft 210 and a bob 214 at an end of the shaft 210 and an elongate member 220 at connected to the shaft 210 at the bob 214. The shaft 210 is cylindrical and is configured to vibrate torsionally about a longitudinal axis 212 through the centre of the shaft 210 at an angular frequency of vibration, ω. As with the vibratory transducer of FIG. 21, the bob 214 has three axial sections that are axially aligned with the axis 212 of the shaft 210, including: i) a first axial section that is frustoconical, where the radius increases linearly across the axial section from an initial radius, that is equal to the shaft radius, to a final radius; ii) a second axial section that is cylindrical and has a radius equal to the final radius of the first axial section; and iii) a third axial section that is conical, where the radius decreases linearly from the radius of the second axial section to zero. The elongate member 220 is cylindrical and has a radius R and is connected to the second axial section of the bob 214. The elongate member 220 has a longitudinal axis 222 that is perpendicular to the axis 212 of the shaft 210 about which the shaft 210 vibrates. A proximal end of the elongate member 220 is offset from the axis 212 of the shaft 210 by a first offset distance R_{O,1} that is equal to a radius of second axial section of the bob 214. A distal end of the elongate member 220 is offset from the axis 212 of the shaft 210 by a second offset distance R_{O,2} that is equal to the sum of the radius of the second axial section of the bob 214 and the length of the elongate member 220. The elongate member 220 is offset from the axis 212 of the shaft 210 along its entire length. The shaft 210 (including the bob 214) and the elongate member 220 are in contact with a fluid 200. In accordance with the techniques of this disclosure, a beneficial level of geometric damping is obtained when the viscoelastic propagation distance of a shear wave in the fluid 200 at the angular frequency of vibration is greater than the radius R of the elongate member 220 and when a Reynolds number of flow of the fluid 200 around the elongate member 220 is low. The offsetting of the elongate member 220 from the axis 212 of the shaft 210 provides increased damping than would have been obtained if the axis 222 of the elongate member 220 was in line with the axis 212 of the shaft 210.

FIG. 23 illustrates a vibratory transducer for use with the techniques of this disclosure. The vibratory transducer comprises a shaft 210 and a bob 214 at an end of the shaft 210 and two elongate members 240a, 240b connected to the shaft 210 at the bob 214. The shaft 210 is cylindrical and is configured to vibrate torsionally about a longitudinal axis 212 through the centre of the shaft 210 at an angular frequency of vibration, ω. As with the vibratory transducer of FIG. 21, the bob 214 has three axial sections that are axially aligned with the axis 212 of the shaft 210, including: i) a first axial section that is frustoconical, where the radius increases linearly across the axial section from an initial radius, that is equal to the shaft radius, to a final radius; ii) a second axial section that is cylindrical and has a radius equal to the final radius of the first axial section; and iii) a third axial section that is conical, where the radius decreases linearly from the radius of the second axial section to zero. Each elongate member 240a, 240b is connected to the second axial section of the bob 214 on diametrically opposite sides of the second axial section of the bob 214. Each elongate member 240a, 240b is conical about an axis 242 that is perpendicular to the axis of the shaft 210 about which the shaft 210 vibrates. Each elongate member 240a, 240b has a maximum radius Rₘₐₓ at a proximal end where it connects to the bob 214. The average radius of an elongate member 240a, 240b along its length is Rₐᵥₑ, which is less than Rₘₐₓ. For each elongate member 240a, 240b, a proximal end is offset from the axis 212 by a first offset distance R_{O,1} that is equal to a radius of the second axial section of the bob 214 and a distal end is offset from the axis 212 by a second offset distance R_{O,2} that is equal to the sum of the radius of the second axial section of the bob 214 and the length of the elongate member 240a, 240b. The whole of each elongate member 240a, 240b (including both ends) is offset from the axis 212 of the shaft 210. The shaft 210 (including the bob 214) and both elongate members 240a, 240b are in contact with a fluid 200. In accordance with the techniques of this disclosure, a beneficial level of geometric damping is obtained when the viscoelastic propagation distance of a shear wave in the fluid 200 at the angular frequency of vibration is greater than Rₐᵥₑ (more preferably greater than Rₘₐₓ) and when a Reynolds number of flow of the fluid 200 around the elongate members 240a, 240b is low. The offsetting of the elongate members 240a, 240b from the axis 212 of the shaft 210 provides increased damping than would have been obtained if each elongate member 240a, 240b had its axis 242 in line with the axis 212 of the shaft 210.

In another configuration in accordance with the techniques of this disclosure, a vibratory transducer includes a shaft and a plurality of elongate members. If the vibratory transducer comprises a bob, then the elongate members may be connected to the vibratory transducer at the bob. Alternatively or additionally, the vibratory transducer may comprise elongate members connected to the vibratory transducer at the shaft. A plurality of elongate members are spaced around the circumference of the shaft or bob, extending outward from the shaft or bob in a wholly radial direction or in a direction with a radial component and an axial component, or in a wholly axial direction (that is not colinear with a longitudinal axis of the shaft/bob). The plurality of elongate members may be distributed evenly around the circumference, which may mitigate or avoid any disturbance in the centre of mass relative to the longitudinal axis, or may be distributed unevenly around the circumference. The plurality of elongate members may be connected to the shaft or bob at the same axial position along the length of the shaft or bob, or may be connected at different axial positions, e.g. in a helical pattern around the exterior surface of the shaft or bob.

If the plurality of elongate members extend from the shaft or bob in a wholly or partially axial direction, then the elongate members may include spacing supports from an outer surface of the shaft or bob to provide a radial offset to the elongate members. Alternatively, the plurality of elongate members may extend from an end of the shaft or bob, such as distributed in a circle around the longitudinal axis and extending from the end of the shaft or bob. The end of the shaft or bob may be flat, curved, conical or have some other profile.

The elongate members may have a width and half width such that geometric damping (monopole behaviour) may take place around the elongate members during vibration of the vibratory transducer. The vibration of the vibratory transducer may be torsional about the longitudinal axis of the shaft.

The vibratory transducer may comprise two or more elongate members, such as three, four, five, six, seven, eight, nine, ten or more elongate members. The elongate members may have constant cross sections along their lengths (such as a cylindrical elongate member or an elongate member having a square/rectangular, rounded square/rectangular (e.g. in the form of a superellipse), triangular or elliptical cross section) or may have varying cross sections shapes or sizes along their lengths, such as i) a cone of linearly decreasing cross-section area with increasing distance from the shaft or bob, or ii) stepped variations in size or shape (e.g. stepped decrements in size) with increasing distance from the shaft or bob.

In a particular configuration, a vibratory transducer comprises a shaft configured for torsional vibration, the shaft having a proximal end at which the vibrations are driven and a distal end. At or close to the distal end of the shaft (e.g. nearer to the distal end than the proximal end, or within a final quarter of the fluid-contacting length of the shaft, or within a final tenth of the fluid-contacting length of the shaft, or within a final twentieth of the fluid-contacting length of the shaft), a plurality of elongate members extend radially outward from the shaft at a common axial position along the length of the shaft. Eight elongate members are distributed evenly around the circumference of the shaft at 45° increments. In another particular configuration, a bob is present at the distal end of the shaft and the eight elongate members extend radially outward from bob. Other configurations comprise more or fewer elongate members, distributed evenly around the shaft/bob or distributed unevenly. For example, one configuration comprises six elongate members distributed evenly around the circumference of the shaft.

In another particular configuration, a vibratory transducer includes a shaft and a plurality of elongate members that are aligned axially with the longitudinal axis of the shaft but are not colinear with the longitudinal axis of the shaft. At a distal end of the shaft there is a bob. The bob has the form of a cylinder that is coaxial with the longitudinal axis of the shaft but has a larger radius than the shaft. The plurality of elongate members extend axially outward from an end of the bob, each connected to the end of the bob at the same radial offset from the longitudinal axis and distributed evenly around the longitudinal axis. The plurality of elongate members comprise eight elongate members that are distributed evenly around the longitudinal axis at 45° increments. The vibratory transducer is configured to vibrate torsionally about the longitudinal axis. Other configurations comprise more or fewer elongate members, distributed evenly around the longitudinal axis or distributed unevenly. For example, one configuration comprises six elongate members distributed evenly around the longitudinal axis.

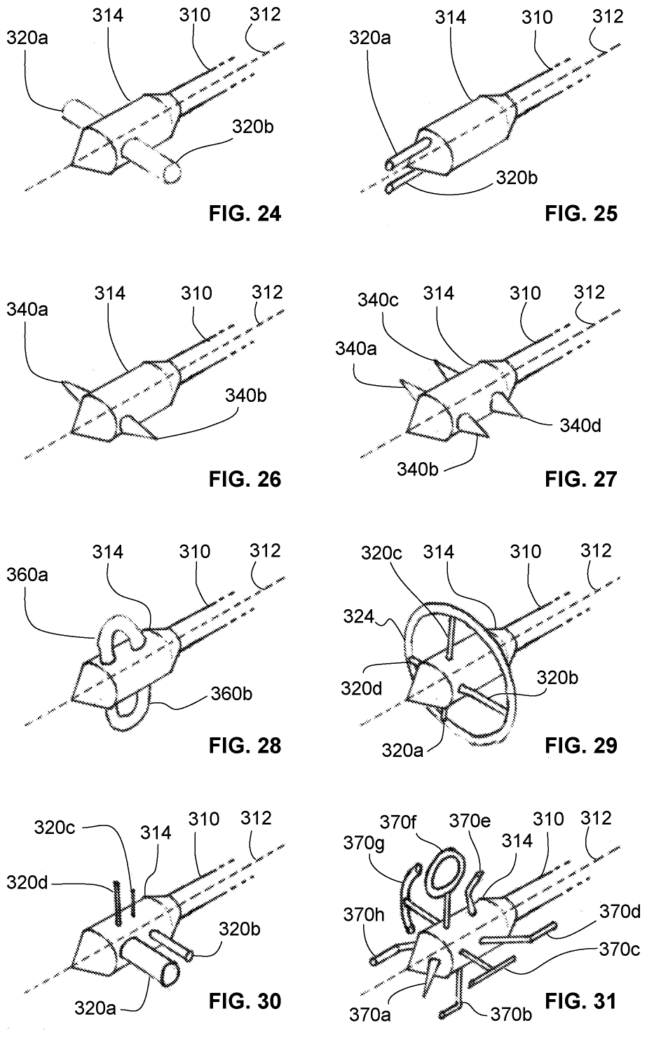

FIGS. 24 to 31 illustrate further vibratory transducers for use with the techniques of this disclosure, in which a shaft 310 is configured to vibrate torsionally about a longitudinal axis 312, the shaft having a bob 314 that has three axial sections that are axially aligned with the axis 312 of the shaft 310, including: i) a first axial section that is frustoconical, where the radius increases linearly across the axial section from an initial radius, that is equal to the shaft radius, to a final radius; ii) a second axial section that is cylindrical and has a radius equal to the final radius of the first axial section; and iii) a third axial section that is conical, where the radius decreases linearly from the radius of the second axial section to zero. In each of FIGS. 24 to 31, a plurality of elongate members are connected to the shaft 310 at the bob 314.

FIG. 24 illustrates a vibratory transducer in which two elongate members 320a, 320b are connected to the second axial section of the bob 314. The two elongate members 320a, 320b are cylindrical and have the same radius that is constant along their lengths. A first of the elongate members 320a is diametrically spaced around the bob 314 from the other elongate member 320b. Each elongate member 320a, 320b extends outwards from the bob 314 in a radial direction that is perpendicular to the axis 312 of the shaft 310.

FIG. 25 illustrates a vibratory transducer in which two elongate members 320a, 320b are connected to the third axial section of the bob 314. Each elongate member 320a, 320b is aligned along a respective axis that is parallel to the axis 312 of the shaft 310 but offset radially from the axis 312 of the shaft 310. The two elongate members 320a, 320b are cylindrical and have the same radius that is constant along their lengths. A first of the elongate members 320a is diametrically spaced around the third axial section of the bob 314 from the other elongate member 320b.

FIG. 26 illustrates a vibratory transducer in which two elongate members 340a, 340b are connected to the second axial section of the bob 314. The two elongate members 340a, 340b are conical. A first of the elongate members 340a is diametrically spaced around the bob 314 from the other elongate member 340b. Each elongate member 340a, 340b extends outwards from the bob 314 in a radial direction that is perpendicular to the axis 312 of the shaft 310. Each elongate member 340a, 340b has a maximum radius at a proximal end, at which it is connected to the second axial section of the bob 314.

FIG. 27 illustrates a vibratory transducer in which four elongate members 340a, 340b, 340c, 340d are connected to the second axial section of the bob 314. The four elongate members 340a, 340b, 340c, 340d are conical. A first of the elongate members 340a is diametrically spaced around the bob 314 from a second elongate member 340b but at the same axial location along the axis 312 as the second elongate member 340b. A third of the elongate members 340c is diametrically spaced around the bob 314 from a fourth elongate member 340d but at the same axial location along the axis 312 as the fourth elongate member 340d. The first and second elongate members 320a, 320b are axially spaced along the axis 312 from the third and fourth elongate members 340c, 340d respectively. Each elongate member 340a, 340b, 340c, 340d has a maximum radius at a proximal end, at which it is connected to the second axial section of the bob 314.

FIG. 28 illustrates a vibratory transducer in which two elongate members 360a, 360b are connected to the second axial section of the bob 314. The two elongate members 360a, 360b each have the form of a semitorus. A first elongate member 360a is curved and connects to the bob 314 at two axially spaced locations and has a circular cross section along the curved axis through elongate member 360a. A second elongate member 360b is curved and connects to the bob 314 at two axially spaced locations that are diametrically spaced around the bob from the locations where the first elongate member 360a connects to the bob 314. The second elongate member also has a circular cross section along the curved axis through elongate member 360b.

FIG. 29 illustrates a vibratory transducer in which four elongate members 320a, 320b, 320c, 320d are connected to the second axial section of the bob 314. Each of the four elongate members 320a, 320b, 320c, 320d are cylindrical with the same constant radius and extend in a radial direction from the bob 314 from locations distributed around a circumference of the second axial section at 90° from neighbouring elongate members. Each distal end of the four elongate members 320a, 320b, 320c, 320d is connected to a fifth elongate member 324 that encircles the bob 314 and has the form of a torus.

FIG. 30 illustrates a vibratory transducer in which four elongate members 320a, 320b, 320c, 320d are connected to the second axial section of the bob 314. The four elongate members 320a, 320b, 320c, 320d are cylindrical but have different radiuses. A first elongate member 320a has a largest radius. A second elongate member 320b has a radius that is smaller than the radius of the first elongate member 320a. A third elongate member 320c has a radius that is smaller than the radius of the second elongate member 320b. A fourth elongate member 320d has a radius that is smaller than the radius of the third elongate member 320c. The first elongate member 320a and second elongate member 320b each extend radially outward from the second axial section of the bob 314 from respective locations that are axially offset from each other but otherwise from the same circumferential location. The third elongate member 320c and second elongate member 320d each extend radially outward from the second axial section of the bob 314 from respective locations that are axially offset from each other but otherwise from the same circumferential location. The first and second elongate members 320a, 320b are spaced around the circumference of the bob 314 from the third and fourth elongate members 320c, 320d by 90°.

FIG. 31 illustrates a vibratory transducer in which eight elongate members 370a, 370b, 370c, 370d, 370e, 370f, 370g, 370h are connected to the bob 314. A first elongate member 370a is connected to the third axial section of the bob 314 and has the form of a cone extending in a direction that oblique to the axis 312 of the shaft 310. A second elongate member 370b is connected to the second axial section of the bob 314 and extends, from its proximal end, radially outward from the bob 314. The second elongate member 370b is not straight-it comprises two straight sections that connect at a right angle to form an ‘L’-shaped member. The distal section of the second elongate member 370b extends in a direction parallel to the axis 312. The third elongate member 370c is connected to the second axial section of the bob 314 and has the form of a ‘T’-shape. A proximal section of the third elongate member 370c is connected to the bob 314 and extends radially outward from the bob 314. A distal section of the third elongate member 370c is connected to a distal end of the proximal section of the third elongate member 370c and extends in a direction parallel to the axis 312, i.e. perpendicular to the direction of the proximal section. The fourth elongate member 370d is connected to the second axial section of the bob 314 and comprises a proximal section and a distal section, the proximal section connected to the bob 314 and extending in an oblique direction relative to the axis 312 of the shaft 310, an end of the distal section connected to a distal end of the proximal section, the distal section extending in a direction parallel to the axis 312. The fifth elongate member 370e is connected to the second axial section of the bob 314. The fifth elongate member 370e is not straight but extends outward from the bob 314 in a broadly radial direction but it has a broadly circular cross section along its length. The sixth elongate member 370f is connected to the second axial section of the bob 314 and extends radially outward from the bob 314. At a distal end of the sixth elongate member 370f, a further elongate member is connected, the further elongate member having the form of a closed loop and specifically a torus. The seventh elongate member 370g is connected to the second axial section of the bob 314 and extends radially outward from the bob 314. At a distal end of the seventh elongate member 370g, a further elongate member is connected, the further elongate member being curved in a circumferential about an arc that is concentric with the circumference of the second axial section of the bob 314. The eighth elongate member 370h is connected to the third axial section of the bob 314 and comprises a proximal section and a distal section, the proximal section connected to the bob 314 and extending outward from the surface of the third axial section in an oblique direction relative to the axis 312 of the shaft 310, an end of the distal section connected to a distal end of the proximal section, the distal section extending in a direction parallel to the axis 312.

In the embodiments discussed above, the elongate members used for geometric damping have circular cross sections, from which a radius is readily determined. The analysis presented above makes use of a radius of an elongate member in determining whether or not the conditions for geometric damping are satisfied. However, the skilled reader will recognise that the techniques of this disclosure are not wholly reliant on the elongate member having a cross section that is exactly circular. A circular cross section may be a preferred embodiment, particularly for torsional vibrations about an axis of the elongate member, since such vibrations would produce only shear waves. However, as discussed above, the offsetting of the elongate member from a vibrational axis may still produce monopole-like behaviour in a wave field in some conditions, i.e. when a Reynolds number is sufficiently low that the flow is laminar. Thus the requirement for circularity of the elongate member is only loose. Elongate members having cross sections that are not exactly circular may still provide geometric damping according to the techniques of this disclosure.

One measure of a shape's circularity is given by the following expression:

$$Circularity = \frac{4 π A}{{p}^{2}} ,$$

where p is the perimeter and A is the area of the shape. For a perfect circle, the circularity by this measure will be equal to 1. For a square, the circularity by this measure will be equal to π/4, which is approximately equal to 0.785. For an equilateral triangle, the circularity will be equal to π\sqrt{3}/9, which is approximately equal to 0.605. For a regular hexagon, the circularity will be equal to π\sqrt{3}/6, which is approximately equal to 0.907. In the present disclosure, a reference to a shape being ‘substantially’ circular is intended to mean a shape that has a circularity by this measure that is in the range 0.75 to 1 inclusive, more preferably in the range 0.8 to 1 inclusive, even more preferably in the range 0.85 to 1 inclusive, even more preferably in the range 0.9 to 1 inclusive, even more preferably in the range 0.92 to 1 inclusive, even more preferably in the range 0.95 to 1 inclusive, even more preferably in the range 0.96 to 1 inclusive even more preferably in the range 0.97 to 1 inclusive, even more preferably in the range 0.98 to 1 inclusive, and even more preferably still in the range 0.99 to 1 inclusive. For non-convex shapes, it may be more appropriate to characterise the circularity by way of a convex perimeter and a convex area, where the convex perimeter the perimeter of the convex hull that encloses the shape and the convex area is the area of the convex hull that encloses the shape.

Thus, in characterising the techniques of this disclosure and considering the requirements to obtain geometric damping, it may be more appropriate to consider a half-width of an elongate member rather than a radius, the elongate member having an axis extending along the length of the elongate member and a cross section that is either constant or varies along the axis, the cross section having a half width that represents the radius in the analysis presented above. The characterising half width used to characterise the geometric damping behaviour of the elongate member at that position along the axis may be a minimum distance from the centroid of the cross sectional area, a maximum distance from the centroid, or a value in between the minimum and maximum distances from the centroid, which may be an average of the minimum and maximum distances, or an average of the distance from the centroid averaged around perimeter of the cross section. Alternatively, the half width may be determined from the perimeter itself of an equivalent circle, or from the convex perimeter if the shape is concave. For example, if the convex perimeter is p, the half width to characterise the shape may be calculated as p/2π. In the case of a circle, this expression would return the radius of the circle. But shapes that are not a perfect circle, this formula would return an equivalent ‘radius’ (i.e. the half width) that would characterise the geometric damping behaviour of the elongate member. As an alternative, a half width to characterise the shape may be based on the convex area of the shape for the equivalent circle, i.e. √(A/π).

In some embodiments, the elongate member has a constant cross section along its length. Therefore a calculation of the half width characterising the geometric damping behaviour of the elongate member can be made at any point along the length. In some other embodiments, the elongate member has a non-constant cross section along its length. The radius or half width may vary along the length of the elongate member. Therefore it may be more appropriate to calculate a characterising radius for the elongate member. Conservatively, if the maximum half width along the length of the elongate member is less than the viscoelastic propagation depth, then geometric damping can be assumed to present. However, as shown in FIG. 4, the deviation from the linear relationship between damping factor and viscosity from linear may remain somewhat gradual even where a radius slightly exceeds the viscoelastic propagation depth. Therefore, taking a less conservative approach, geometric damping may still be achieved if an average measure of the non-constant half width is less than the viscoelastic propagation depth. Such an average may be calculated from an average of the half width along the length of the elongate member, such as an arithmetic mean of the halfwidth along the length of the elongate member, or by calculating an average from the volume and surface area of the elongate member by the expression:

$$\frac{2 V}{A} ,$$

where V is the volume of the elongate member and A is the surface area; these quantities may be volumes and areas of convex hulls of the elongate member in the case of non-convex forms.

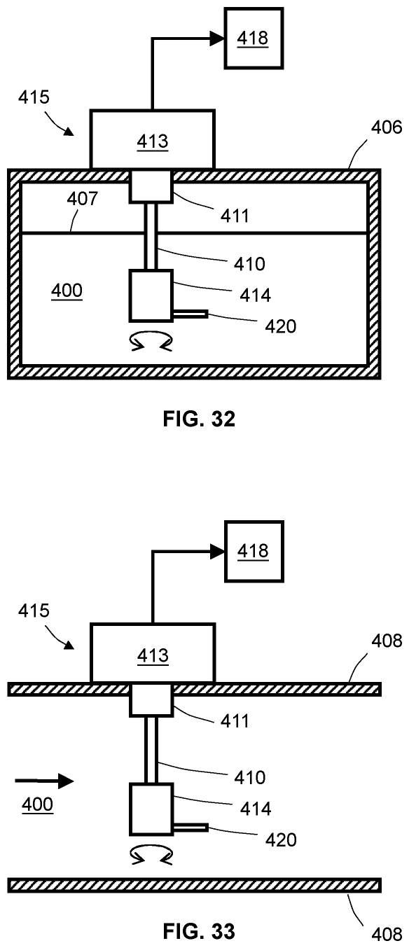

FIG. 32 illustrates schematically an example apparatus for analysing a fluid using one or more techniques of this disclosure. The apparatus comprises a vibratory transducer 415 in a fluid sample 400. The fluid sample 400 in this instance is a fixed volume of generally stationary fluid in a chamber 406, the fluid having a free surface 407, and portions of the viscosity transducers piercing the free surface 407 from above.

While the chamber 406 is drawn in FIG. 32 having a closed top wall, as may be necessary if its contents are pressurized, the chamber may equally be open from above. The fluid sample may therefore be at atmospheric pressure. In such a configuration, the vibratory transducer may be located above the chamber 407 such that at least portions of the vibratory transducer extend into the upper opening of the chamber 407 and contact the fluid sample 405 instead.

The vibratory transducer 415 includes a vibrating element configured to oscillate in a torsional mode. The vibrating element is immersed in the fluid and the viscosity is determined by correlation with the damping experienced by the element, i.e. the Q factor. In particular, the vibratory transducer comprises a sensor mounting 413, a semi-rigid connection member 411, a shaft 410 and a bob or ‘sense element’ 414. The shaft 410 and the bob 414 are driven to vibrate torsionally about a longitudinal axis of the shaft 410 with an angular frequency ω. The bob 414 is of relatively large mass and the shaft 410 and bob 414 are formed, at least substantially and possibly entirely, of a metal material such as a stainless steel. The bob 414 and shaft 410 both have a circular cross-section, other than the presence of an elongate member 420 in accordance with the techniques of this disclosure extending radially outwards from the circumference of the bob 414. The whole of the elongate member 420 is offset from the longitudinal axis of the shaft 410.

The bob 414 and the elongate member 420 are exposed to the viscous effect of the fluid in the sample 400. Increasing viscosity of the fluid causes an increased damping of the vibration in the sensor, resulting in a measurable reduced vibrational efficiency of the system. A measurement of a quantity indicative of a degree of damping in the system, such as a Q factor or loss factor, is provided from the vibratory transducer to a processor 418 configured to process the measurement and determinate a physical property of the fluid sample 400, such as a viscosity. The processor 418 may be integral with the vibratory transducer 415 or connected to the vibratory transducer via a data interface.

FIG. 33 illustrates schematically a further example apparatus for analysing a fluid using one or more techniques of this disclosure. The apparatus includes the same vibratory transducer 415 as in FIG. 32, but in this instance the fluid sample 400 is flowing in a conduit 418 at an upstream average speed of, e.g. 1 m/s. The vibratory transducer 415 extends from above through the wall of the conduit 408, the portions extending through the wall of the conduit 408 being in contact with the fluid sample 400 as it flows past the vibratory transducer 415. As in FIG. 32, the vibratory transducer 415 includes an elongate member 420 in accordance with the techniques of this disclosure extending radially outwards from the circumference of the bob 414. The whole of the elongate member 420 is offset from the longitudinal axis of the shaft 410.

It is emphasised that, while FIG. 32 and FIG. 33 each illustrate a vibratory transducer comprising a bob 414, the techniques of this disclosure are not limited to vibratory transducers comprising bobs.

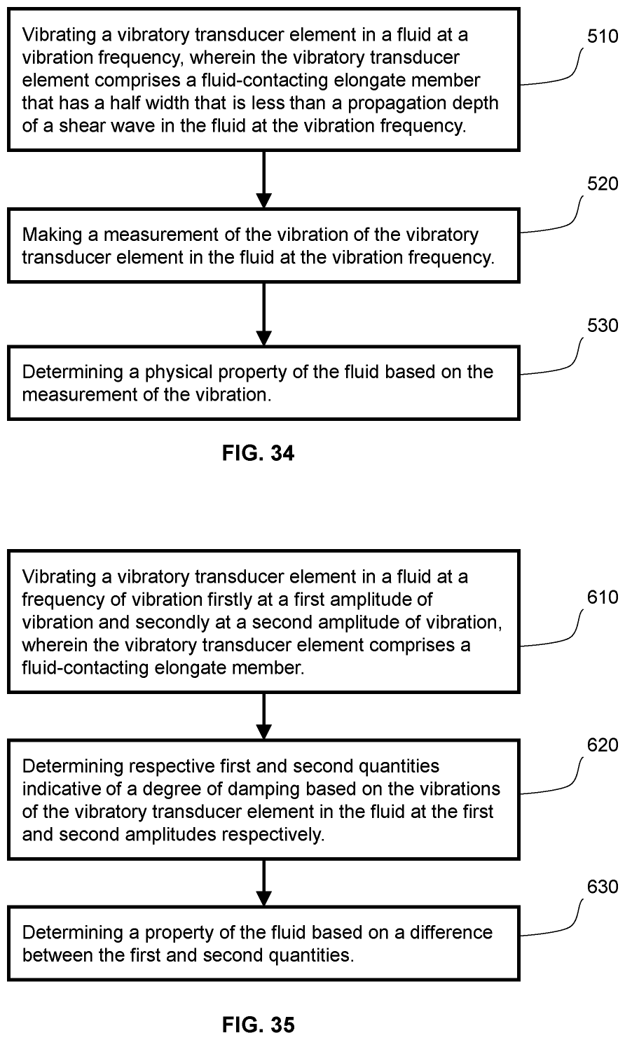

FIG. 34 illustrates a flow chart of a method in accordance with the techniques of this disclosure. The method includes a first step 510 of vibrating a vibratory transducer element in a fluid at a vibration frequency, wherein the vibratory transducer element comprises a fluid-contacting elongate member that has a half width that is less than a propagation depth of a shear wave in the fluid at the vibration frequency. The method includes a second step 520 of making a measurement of the vibration of the vibratory transducer element in the fluid at the vibration frequency. The method includes a third step 530 of determining a physical property of the fluid based on the measurement of the vibration. In accordance with the techniques of this disclosure, determining the physical property of the fluid based on the measurement of vibration may comprise determining one or more of: a viscosity, a viscoelasticity, a density, a fluid stiffness, a loss tangent, a storage modulus, a loss modulus, and a yield stress.

FIG. 35 illustrates a flow chart of a further method in accordance with the techniques of this disclosure. The method includes a first step 610 of vibrating a vibratory transducer element in a fluid at a frequency of vibration firstly at a first amplitude of vibration and secondly at a second amplitude of vibration, wherein the vibratory transducer element comprises a fluid-contacting elongate member. The method includes a second step 620 of determining respective first and second quantities indicative of a degree of damping based on the vibrations of the vibratory transducer element in the fluid at the first and second amplitudes respectively, wherein the first and second quantities may in some embodiments be Q factors. The method includes a third step 630 of determining a property of the fluid based on a difference between the first and second quantities, which may be, for example: i) determining a dipolarity of the wave field around the vibratory transducer at the frequency of vibration based on the difference between the first and second quantities, ii) determining a Reynolds number of the fluid based on the difference between the first and second quantities, or iii) determining, based on the difference between the first and second quantities, a velocity of vibration relative to the fluid, a viscosity of the fluid, or a density of the fluid.

In some embodiments, the method described in FIG. 35 is combined with the method described in FIG. 34, the method of FIG. 35 used to determine a dipolarity of the wave field around the vibratory transducer to confirm or used to determine a Reynolds number of the fluid, with the purpose of determine or confirm if geometric damping is present.

Vibratory transducers in accordance with the techniques described herein may be used to determine a physical property of a fluid by vibrating the vibrating transducer in a fluid at a vibration frequency and determining a quantity indicative of a degree of damping based on the vibration. For example, to make a measurement of the viscosity, the Q factor of the vibration can be determined. The Q factor is a dimensionless parameter that indicates the level of damping of a resonator, wherein the level of damping is a function of the viscosity. In particular, it indicates the degree to which a resonator is underdamped. On a plot of frequency response, a high Q factor provides a high and narrow peak at the resonant frequency whereas a low Q factor provides a low and wide peak. Due to the change in width of the peak with damping, the Q factor can be defined as the ratio of the resonant frequency to the resonant bandwidth:

$$Q = \frac{{ω}_{R}}{Δ ω} \qquad ( Equation 43 )$$

wherein ω_{R} is the resonant frequency in radians per second and Aw is the Full Width at Half Maximum (FWHM), the bandwidth over which the power of the vibration is greater than half of the maximum (or equivalently the amplitude of vibration is greater than the maximum amplitude at resonance divided by √2), i.e. the bandwidth between the 3 dB points. The fluid viscosity is a function of the Q factor. Where geometric damping does not apply, the fluid viscosity is inversely proportional to the square of the Q factor and any constant of proportionality needed to compute the value of the viscosity measurement can be obtained by calibration with reference fluids of known viscosity. Where geometric damping applies, the fluid viscosity is inversely proportional to the Q factor. The fluid viscosity may be determined under geometric damping conditions based on a quantity that is inversely proportional to a measurement or estimate of the Q factor (assuming mechanical damping of the vibrational system is small or negligible relative to damping provided by the fluid loading).

It should be noted that the measurement of viscosity at or corresponding to a frequency of vibration may comprise making amplitude measurements at more than one frequency to estimate the Q factor but a single viscosity measurement is obtained at a frequency corresponding to the or a resonant frequency. For example, the bandwidth can be determined based on the frequencies required to cause the amplitude to drop to a factor of 1/√2 of the maximum amplitude at resonance. As a non-limiting example, the frequencies required to cause the amplitude to drop to a factor of 1/√2 of the maximum amplitude at resonance may be determined by performing a frequency sweep around the resonant frequency, but the skilled reader will recognize that the 3 dB point frequencies can be identified by various other techniques.

Another approach to determining the Q factor is to measure the amplitude of vibration at a series of frequencies around the resonant frequency and fit a parabola by the method of least squares to the frequency and amplitude values (or logarithms thereof). The 3 dB points can then be obtained as solutions to a quadratic equation based on the parabola of best fit to the measurements.

Another approach to determining the Q factor is by logarithmic decrement. By ceasing to drive the transducer and measuring the decay of vibrations, the Q factor may be determined by monitoring time series of the vibrations and determining the natural logarithm of the ratio of two successive peaks, A₁ and A₂, by the following expression:

$$Q = \frac{1}{2} \sqrt{1 + {( \frac{2 π}{ln ( \frac{{A}_{1}}{{A}_{2}} )} )}^{2}} . \qquad ( Equation 44 )$$

As an alternative to determining a Q factor, the techniques of this disclosure encompass determining a ‘loss factor’, wherein the loss factor is the reciprocal of the Q factor and may be determined by corresponding techniques to those set out above in respect of the Q factor.

In the techniques described herein, where it is necessary to determine a location of an ‘end’ of an elongate member, the location of the end of the elongate member should be considered to be the location of the centroid of the cross-section area of the elongate member at that end.

While some of the embodiments described in this disclosure relate to the determining of properties of a fluid, the skilled reader will recognise that the geometric damping techniques described herein may have wider application.

For example, geometric damping may be employed for the deliberate modification or control of a damping characteristic of any item that is or will be vibrating in a fluid. An example may be a shaft that vibrates in a fluid, as is frequently encountered in industry. A damping characteristic may be controlled by providing the shaft with one or more elongate members in accordance with the techniques of this disclosure. If the one or more elongate members are sized correctly relative to a propagation depth of a shear wave in the fluid at a frequency of vibration of the shaft, then geometric damping may arise. Moreover, if the one or more elongate members are spaced from a longitudinal axis of the shaft, then amplification of damping effects may be obtained, while geometric damping may be retained if the fluid flow regime permits at least the recovery of a partial monopole wave field. An appropriate number of elongate members having an appropriate geometry may be provided to the shaft in accordance with the techniques of this disclosure to provide a desired damping characteristic that is independent of fluid properties, provided the geometric damping criterion is satisfied. In some cases the vibration may be unwanted, and thus a goal of controlling the damping behaviour is to provide a relatively high degree of damping such that vibration is attenuated.

A method of controlling a damping behaviour of a shaft that is configured to vibrate in a fluid comprises: providing a shaft configured to vibrate at a vibration frequency, the shaft having a longitudinal axis; and providing an elongate member connected to the shaft but not collinear with the longitudinal axis of the shaft, the elongate member characterised by a width, a half width that is equal to half of the width, and a length that is greater than the width, the elongate member and at least a portion of the shaft configured to vibrate in the fluid at the vibration frequency, wherein the half width of the elongate member is less than a propagation depth of a shear wave in the fluid at the vibration frequency.

As discussed previously, the propagation depth may be a distance over which an amplitude of a shear wave propagating in the fluid at the vibration frequency is reduced by a factor of 1/e and/or may be a viscoelastic propagation depth in accordance with (Equation 1).

Such an elongate member may be any of the elongate members described herein, including any of the elongate members illustrated in FIGS. 7 to 31. In some embodiments, the elongate member has a first end and a second end, wherein one or both of the first and second ends is spaced from the longitudinal axis of the shaft by an offset distance that is greater than the half width of the elongate member.

The elongate member may be provided such that, during vibration of the shaft at the vibration frequency, the flow of fluid around the elongate member is laminar flow. This may affect choice of dimensions of an elongate member in order to obtain laminar flow around the elongate member during vibration of the shaft.

In some embodiments, the elongate member is provided such that during vibration of the shaft in the fluid at the vibration frequency, a Reynolds number, Re, of fluid flow around the elongate member is less than one, wherein the Reynolds number is given by:

$$Re = \frac{2 R v ρ}{μ} ,$$

wherein μ is a viscosity of the fluid, ρ is a density of the fluid, R is the half width of the elongate member, and v is a maximum velocity of the elongate member relative to the fluid during vibration of the shaft. A choice of length and half width of the elongate member will affect a Reynolds number and therefore the Reynolds number will represent a constraint on dimensions of the elongate member.

If, for example, the dimensions of the elongate member subject to the Reynolds number criterion do not provide a desired level of damping, the shaft may be provided with a plurality of elongate members connected to the shaft that are each not collinear with the longitudinal axis of the shaft, each having a half width that is less than the propagation depth of a shear wave in the fluid at the vibration frequency. In this way, the damping characteristic of the shaft is affected by the combined effects of the plurality of elongate members.

The skilled reader will further recognise that these techniques are not limited to the provision of elongate members on vibrating shafts but may encompass the provision of one or more elongate members on any vibrating object, such as a plate configured to vibrate in an in-plane direction at a vibration frequency to generate shear waves. The provision of one or more elongate members connected to the plate (e.g. extending perpendicularly or obliquely outward from the plate) that have a half width that is below a propagation depth of a shear wave in the fluid at the vibration frequency may cause geometric damping as described herein to arise.

The skilled reader will further appreciate that the various illustrative logical blocks, configurations, modules, circuits, and algorithm steps described in connection with the embodiments disclosed herein may be implemented as electronic hardware, computer software, or combinations of both. To clearly illustrate this interchangeability of hardware and software, various illustrative components, blocks, configurations, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The steps of a method or algorithm described in connection with the embodiments disclosed herein may be embodied directly in hardware, in a software module executed by a processor, or in a combination of the two. A software module may reside in random access memory (RAM), flash memory, read-only memory (ROM), programmable read-only memory (PROM), erasable programmable read-only memory (EPROM), electrically erasable programmable read-only memory (EEPROM), registers, hard disk, a removable disk, a compact disc read-only memory (CD-ROM), or any other form of storage medium known in the art. An exemplary storage medium is coupled to the processor such that the processor can read information from, and write information to, the storage medium. In the alternative, the storage medium may be integral to the processor. The processor and the storage medium may reside in an application-specific integrated circuit (ASIC). The ASIC may reside in a computing device or a user terminal. In the alternative, the processor and the storage medium may reside as discrete components in a computing device or user terminal.

The previous description of the disclosed embodiments is provided to enable any person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as defined by the following claims.

1. A method of determining a physical property of a fluid, the method comprising:
vibrating a vibratory transducer element in a fluid at a vibration frequency, wherein the vibratory transducer element comprises a fluid-contacting elongate member characterised by a width, a half width that is equal to half of the width, and a length that is greater than the width, wherein the half width is less than a propagation depth of a shear wave in the fluid at the vibration frequency;
making a measurement of the vibration of the vibratory transducer element in the fluid at the vibration frequency; and
determining a physical property of the fluid based on the measurement of the vibration.

2. The method of claim 1, wherein determining the physical property of the fluid based on the measurement of vibration comprises determining one or more of: a viscosity, a viscoelasticity, a density, a fluid stiffness, a loss tangent, a storage modulus, a loss modulus, and a yield stress.

3. The method of claim 2, wherein making the measurement of the vibration comprises determining a quantity indicative of a degree of damping of the vibratory transducer element in the fluid at the vibration frequency, and wherein determining the physical property of the fluid based on the measurement of the vibration comprises determining a viscosity of the fluid based on the quantity indicative of the degree of damping.

4. The method of claim 2, wherein making the measurement of the vibration comprises determining a first quantity indicative of a degree of damping of the vibratory transducer element in the fluid at the vibration frequency, wherein the method further comprising vibrating the vibratory transducer element in the fluid at a further vibration frequency and determining a second quantity indicative of a degree of damping of the vibratory transducer element in the fluid at the further vibration frequency, wherein determining the physical property of the fluid based on the measurement of the vibration comprises determining a viscoelasticity of the fluid based on the quantities indicative of the degree of damping at the vibration frequency and at the further vibration frequency.

5. The method of claim 2, wherein making the measurement of the vibration comprises determining a resonant frequency of the vibratory transducer element in the fluid, wherein determining the physical property of the fluid based on the measurement of the vibration comprises determining a density of the fluid based on the resonant frequency.

6. The method of any preceding claim, wherein the propagation depth is a distance over which an amplitude of a shear wave propagating in the fluid at the vibration frequency is reduced by a factor of 1/e, wherein e is the base of natural logarithms.

7. The method of any preceding claim, wherein the propagation depth of a shear wave propagating in the fluid at the vibration frequency is given by the expression:
$$\frac{1}{sin \frac{Δ}{2}} \sqrt{\frac{μ}{ρ ω sin Δ}} ,$$
wherein μ is a viscosity of the fluid, ρ is a density of the fluid, ω is the angular frequency of vibration, and Δ varies between 0 and π/2 and is defined by the loss tangent, tan Δ, and wherein tan Δ is equal to the following expression, in which G′ is a storage modulus of the fluid:
$$\frac{μ ω}{{G}^{′}} .$$

8. The method of any preceding claim, wherein the half width of the elongate member is less than 50% of the propagation depth.

9. The method of any preceding claim, wherein the vibratory transducer element comprises a shaft that has a longitudinal axis, wherein the elongate member is connected to the shaft and wherein the elongate member is not collinear with the longitudinal axis of the shaft.

10. The method of claim 9, wherein, during vibration of the vibratory transducer element at the vibration frequency, the flow of fluid around the elongate member is laminar flow.

11. The method of claim 9 or claim 10, wherein the elongate member has a first end and a second end, wherein one or both of the first and second ends is spaced from the longitudinal axis of the shaft by an offset distance that is greater than the half width of the elongate member.

12. The method of any of claims 9 to 11, wherein, during vibration of the vibratory transducer element in the fluid at the vibration frequency, a Reynolds number, Re, of fluid flow around the elongate member is less than 1000, preferably less than 100, more preferably less than 10, even more preferably less than 1, wherein the Reynolds number is given by
$$Re = \frac{2 R v ρ}{μ} ,$$
wherein μ is a viscosity of the fluid, ρ is a density of the fluid, R is the half width of the elongate member, and v is a maximum velocity of the elongate member relative to the fluid during vibration of the vibratory transducer.

13. The method of any of claims 9 to 12, wherein the shaft comprises a bob and the elongate member is connected to the shaft at the bob.

14. The method of any of claims 9 to 13, wherein the vibratory transducer element comprises a plurality of elongate members connected to the shaft that are each not collinear with the longitudinal axis of the shaft, each having a half width that is less than the propagation depth of a shear wave in the fluid at the vibration frequency.

15. The method of claim 14, wherein the half width of a first elongate member of the plurality of elongate members is different from the half width of a second elongate member of the plurality of elongate members.

16. The method of any preceding claim, wherein, along at least 50% of its length, the elongate member has a cross section that has a circularity in the range 0.75 to 1, wherein the circularity of a cross section is calculated by:
$$\frac{4 π A}{{p}^{2}} ,$$
where A is the convex area of the cross section and p is the convex perimeter of the cross section.

17. The method of any preceding claim, wherein the elongate member has a constant cross section along at least 90% of its length or has a constant cross section along no more than 10% of its length.

18. The method of any preceding claim, wherein the elongate member is straight or non-straight or comprises one of: a circular cylinder, a cone, a frustrum of a cone, a torus, and an arcuate portion of a torus.

19. The method of any preceding claim, wherein vibrating the vibratory transducer element comprises vibrating the vibratory transducer element with an oscillatory rotational motion and/or an oscillatory rectilinear motion and/or an oscillatory curvilinear motion.

20. The method of claim 19, wherein the elongate member is straight and wherein vibrating the transducer element comprises vibrating the elongate member with an oscillatory rotational motion about an axis along the length of the elongate member.

21. The method of any preceding claim, wherein the length of the elongate member is greater than twice the width of the elongate member.

22. The method of any preceding claim, wherein the half width of the elongate member is greater than 0.5 mm, wherein a viscosity of the fluid is greater than 100 Pas, wherein a density of the fluid is between 500 kg/m³ and 1500 kg/m³, and wherein the frequency of vibration is less than 10 kHz.

23. A device for determining a physical property of a fluid, the device comprising:
a vibratory transducer that includes a shaft configured to vibrate at a vibration frequency, the shaft having a longitudinal axis, wherein the vibratory transducer further includes an elongate member connected to the shaft but not collinear with the longitudinal axis of the shaft, the elongate member characterised by a width, a half width that is equal to half of the width, and a length that is greater than the width,
wherein the device is configured to determine a physical property of a fluid by vibrating the shaft in the fluid at the vibration frequency while the elongate member is in contact with the fluid, wherein, at the vibration frequency, the half width of the elongate member is less than a propagation depth of a shear wave in the fluid,
wherein the device is configured to make a measurement of the vibration of the vibratory transducer in the fluid at the vibration frequency and determine a physical property of the fluid based on the measurement of the vibration at the vibration frequency.

24. The device of claim 23, wherein at least a portion of the elongate member is offset from the longitudinal axis by an offset distance that is greater than the half width of the elongate member.

25. The device of claim 24, wherein the elongate member has a first end and a second end, wherein one or both of the first and second ends is spaced from the longitudinal axis of the shaft by an offset distance that is greater than the half width.

26. The device of any of claims 23 to 25, wherein, along at least 50% of its length, the elongate member has a cross section that has a circularity in the range 0.75 to 1, wherein the circularity of a cross section is calculated by:
$$\frac{4 π A}{{p}^{2}} ,$$
where A is the convex area of the cross section and p is the convex perimeter of the cross section.

27. The device of any of claims 23 to 26, wherein the elongate member has a constant cross section along at least 90% of its length or along no more than 10% of its length.

28. The device of any of claims 23 to 27, wherein the elongate member is straight or non-straight or comprises one of: a circular cylinder, a cone, a frustrum of a cone, a torus, and an arcuate portion of a torus.

29. The device of any of claims 23 to 28, wherein the length of the elongate member is greater than twice the width of the elongate member.

30. The device of any of claims 23 to 29, wherein the half width of the elongate member is greater than 0.5 mm, and/or greater than 1 mm, and/or greater than 2 mm, and/or greater than 5 mm, and/or greater than 10 mm, and/or greater than 20 mm, and/or greater than 50 mm.

31. The device of any of claims 23 to 30, comprising a plurality of elongate members connected to the shaft, each having a respective width, half width and length.

32. The device of claim 31, wherein the half width of a first elongate member of the plurality of elongate members is different from the half width of a second elongate member of the plurality of elongate members.

33. The device of any of claims 23 to 32, wherein the shaft is configured to vibrate torsionally about its longitudinal axis, and/or longitudinally along its longitudinal axis and/or transversely to its longitudinal axis.

34. The device of any of claims 23 to 33, wherein the shaft comprises a bob and the elongate member is connected to the shaft at the bob.

35. A method of determining a property of a fluid, the method comprising:
vibrating a vibratory transducer element in a fluid at a frequency of vibration firstly at a first amplitude of vibration and secondly at a second amplitude of vibration, wherein the vibratory transducer element comprises a fluid-contacting elongate member characterised by a width, a half width that is equal to half of the width, and a length that is greater than the width;
determining a first quantity indicative of a degree of damping based on the vibration of the vibratory transducer element in the fluid at the first amplitude;
determining a second quantity indicative of a degree of damping based on the vibration of the vibratory transducer element in the fluid at the second amplitude;
determining a property of the fluid based on a difference between the first and second quantities.

36. The method of claim 35, wherein determining the property of the fluid comprises determining, based on the difference between the first and second quantities, whether or not the half width of the elongate member is less than the propagation depth of a shear wave in the fluid at the frequency of vibration and/or a degree to which the half width of the elongate member is less than the propagation depth of a shear wave in the fluid at the frequency of vibration.

37. The method of claim 35 or claim 36, wherein determining the property of the fluid comprises determining a Reynolds number of the fluid based on the difference between the first and second quantities.

38. The method of any of claims 35 to 37, wherein determining the property of the fluid comprises determining, based on the difference between the first and second quantities, a velocity of vibration relative to the fluid, a viscosity of the fluid, or a density of the fluid.

39. The method of any of claims 35 to 38, wherein determining the first and second quantities comprises determining first and second Q factors.

40. The method of any of claims 35 to 39, wherein the determined property of the fluid is a property of the flow of the fluid due to the vibration of the vibratory transducer element in the fluid at the frequency of vibration for one or both of the first and second amplitudes.

41. The method of any of claims 35 to 40, further comprising performing a method 5 according to any of claims 1 to 22.

42. The method of any of claims 35 to 41, wherein the method is performed using a device according to any of claims 23 to 34.
